(19) United States
(12) Patent Application Publication
(10) Pub. No.: US 2003/0228623 A1
(43) Pub. Date: Dec. 11, 2003

(54) Methods for generating, selecting, and identifying compounds which bind a target molecule

(76) Inventors: Charles R. Cantor, Del Mar, CA (US); Gabriel O. Reznik, Boston, MA (US); Natalia E. Broude, Natick, MA (US)

Correspondence Address: CLARK & ELBING LLP, 101 FEDERAL STREET, BOSTON, MA, 02110, US

(21) Appl. No.: 10/256,551

(22) Filed: Sep. 27, 2002

Related U.S. Application Data
(60) Provisional application No. 60/325,874, filed on Sep. 28, 2001.
(60) Provisional application No. 60/373,518, filed on Apr. 18, 2002.

(51) Int. Cl. G01N 33/53; G01N 33/567; C12N 7/00; C12N 1/21

(52) U.S. Cl. 435/7.1; 435/7.2; 435/252.33; 435/235.1

(57) ABSTRACT

In general, the invention provides novel methods for generating, selecting, and displaying small molecules on the surface of a virus or cell. The viruses or cells expressing these small molecules may be assayed to select the small molecules that bind a target molecule.

### CROSS REFERENCE TO RELATED APPLICATIONS

This application claims the benefit of the filing dates of U.S. Ser. No. 60/325,874, filed Sep. 28, 2001, and U.S. Ser. No. 60/373,518, filed Apr. 18, 2002, both hereby incorporated by reference.

### BACKGROUND OF THE INVENTION

In general, the invention features novel methods for the generation, selection, and identification of compounds (e.g., small molecules) on the surface of viruses or cells that bind a biological molecule target of interest (e.g., a cell, virus, molecule, or organelle).

Many current drug development paradigms require access to large libraries of potential drug molecules. For example, libraries containing as many as a million compounds are commonly used. These libraries are typically composed of compounds isolated from natural sources (e.g., cell extracts) or generated using combinatorial chemistry. Significant time and effort is required to obtain and screen these libraries to isolate compounds that bind a particular target. Once these candidate drug products are isolated, they must often be optimized using labor-intensive medicinal chemistry methods to increase their affinity for the target molecule.

Recently, in vitro evolution methods have been developed that show great promise in identifying nucleic acids or unmodified proteins with high affinity for a target of interest. These methods include display strategies, such as ribosome display and phage display, which allow multiple rounds of selection to be performed to isolate candidate compounds with high affinity. However, a common limitation of these evolution methods is that they are restricted to relatively large, unmodified nucleic acids and proteins.

Given that most drug products are small compounds (e.g., compounds with molecular weights less than 2,000 daltons) and typically contain functional groups not found in proteins or nucleic acids or not readily amenable to chemical synthesis, new methods are needed that can be used for the production of small molecules containing a variety of complex functional groups. Desirably, these methods may be used to generate and assay novel compounds as well as analyze naturally-occurring molecules to select those that bind a particular target. In addition, these methods are preferably amenable to repeated selection that allows the isolation of candidate drug products with the greatest affinity (e.g., submicromolar affinity) for the target. Additionally, it would be highly desirable to develop methods that may be used to simultaneously analyze different classes of candidate compounds, for example, compounds as diverse as lipids and carbohydrates, and different types of target molecules, for example, proteins, carbohydrates, nucleic acids, small molecules, and infectious agents.

### SUMMARY OF THE INVENTION

The present invention provides novel methods for the rapid production of diverse populations of selectable compounds (e.g., small molecules), as well as the ready selection and identification from such populations of small molecules attached to display peptides that bind target molecules or have desired activities (e.g., antibiotic activity). The present methods exploit cellular processes to generate and display small molecules on the surface of viruses or cells (e.g., bacteria or yeast cells), followed by the selection of those viruses or cells that display binding partners for desired target molecules. In preferred methods, the viruses and cells contain within themselves, either in their genome or in artificial DNA inserts (e.g. plasmids, cosmids, or yeast artificial chromosomes), nucleic acids that encode the proteins responsible for the production of the molecules displayed on their surface. In this case, the selection of a small molecule also yields the genetic information that encodes its design. A portion of, or the entire, selected small molecule may be recovered from the host virus or cell. For example, the small molecule can be chemically or enzymatically cleaved from the display peptide. If desired, recovered compounds may then be identified using standard methods, such as mass spectrometry or NMR. These compounds have a variety of uses including, for example, development of drug products and the study of binding interactions between the compounds and their target molecules.

Accordingly, in a first aspect, the invention provides a method for generating and selecting a small molecule, which binds a target molecule. The method involves expressing in a population of cells a protein fusion that includes a viral surface protein covalently linked to a display peptide. The protein fusion is expressed in the cells prior to, concurrent with, or after the cells are infected with a virus. The expression of the protein fusion is carried out under conditions that allow the display peptide to be modified in the cells with a small molecule and allow the display of the protein fusion on the surface of viruses released from the infected cells. The viruses are contacted with the target molecule, and the viruses which bind the target molecule are selected, as those which display small molecule binding moieties. In this method, the small molecule (i) is covalently bound to a side-chain of an amino acid in the display peptide, (ii) has an unnatural amino acid, (iii) has a molecular weight less than 4,000 daltons and has either an unnatural amino acid or a moiety other than an amino acid, or (iv) has a molecular weight less than 2,000 daltons. In some embodiments, the small molecule is not biotin. In various embodiments, the selected viruses are used to infect additional cells, thereby generating additional viruses which display the desired small molecules. By repeating this process of selection and cell infection to produce identical copies of the selected viruses, the population of viruses may be optionally enriched with viruses that display a small molecule which has a higher affinity for a target.

The invention also provides a related method for selecting compounds which bind target molecules. In this method, candidate compounds produced by cells are added to a display peptide component of a protein fusion after the display peptide is translated. The protein fusion is expressed in the cells prior to, concurrent with, or after the cells are infected with a virus. These posttranslationally modified peptides are displayed on the surface of viruses released from the infected cells that produce the candidate compounds, and the viruses are then assayed to determine if they display candidate compounds (i.e., posttranslational modifications) that bind the target molecule.

In one particular such method, a protein fusion that includes a surface protein covalently linked to a display peptide is expressed in a population of cells, under conditions that allow the posttranslational modification in the cells of the display peptide and the display of the protein fusion on the surface of viruses released from the cells. The viruses are contacted with the target molecule, and the viruses which bind the target molecule are selected, as those which display a desired posttranslational modification. In some embodiments, the posttranslational modification is not biotin. In various embodiments, the viruses are amplified by cell infection to produce identical copies of the selected viruses. By repeating this process of selection and cell infection to produce identical copies of the selected viruses, the population of viruses may be optionally enriched with viruses that display a posttranslational modification which has a higher affinity for a target.

In preferred embodiments of each of the above methods, the process of selection and cell growth is repeated one or more times, and/or a compound (e.g., part or all of a posttranslational modification or small molecule) is recovered from the selected viruses. In this manner, the population of viruses is enriched with viruses that display a small molecule which has a higher affinity for a target. Preferred viruses include filamentous and non-filamentous bacteriophage (such as M13, fl, and fd). A bacteriophage may be used to infect a variety of bacteria, such as Escherichia (e.g., E. coli) or Salmonella. Preferably, the surface protein is a viral coat protein (e.g, pIII or pVIII). In other preferred embodiments, one or more nucleic acids encoding a protein or all of the proteins required for the synthesis of the displayed small molecule or posttranslational modification are contained in the genome of the virus. In still other preferred embodiments, the viruses are used to infect other cells to generate additional viruses that display a selected small molecule or posttranslational modification, thereby producing an essentially unlimited supply of the selected compound.

The selection methods of the present invention may also be performed by displaying small molecules or posttranslational modifications on the surface of cells. In one such aspect, the invention features a method that involves expressing in a population of cells a protein fusion that includes a surface protein covalently linked to a display peptide (e.g., a population of cells capable of surface displaying a variety of different molecules). The expression is carried out under conditions that allow the display peptide to be modified in the cells with a small molecules and the display of the protein fusion on the surface of the cells. The cells are contacted with the target molecule, and the cells which bind the target molecule are selected, as those which display a desired small molecule binder. In this method, the small molecule (i) is covalently attached to a side-chain of an amino acid in the display peptide, (ii) has an unnatural amino acid, (iii) has a molecular weight less than 4,000 daltons and has either an unnatural amino acid or a moiety other than an amino acid, or (iv) has a molecular weight less than 2,000 daltons. In some embodiments, the small molecule is not biotin. In preferred embodiments, the selected cells are cultured under conditions that permit cell proliferation, thereby generating additional cells which express the desired small molecules. By repeating this process of selection and cell growth, the population of cells may optionally be enriched with cells that display a small molecule which has a higher affinity for a target.

In a related aspect, the invention also features a method for generating and selecting a posttranslational modification which binds a target molecule. The method involves expressing in a population of cells a protein fusion that includes a surface protein covalently linked to a display peptide for a posttranslational modification. The expression is carried out under conditions that allow posttranslational modification in the cells of the display peptide and the display of the protein fusion on the surface of the cells. The cells are contacted with the target molecule, and the cells which bind the target molecule are selected, as those which display a desired posttranslational modification. In some embodiments, the posttranslational modification is not biotin. In preferred embodiments, the selected cells are cultured under conditions that permit cell proliferation, thereby generating additional cells which express desired posttranslational modifications. By repeating this process of selection and cell growth, the population of cells may optionally be enriched with cells that display a posttranslational modification which has a higher affinity for a target.

In preferred embodiments of each of the above methods that utilize populations of cells, the process of selection and cell growth is repeated one or more times, and/or a compound (e.g., part or all of a posttranslational modification or small molecule) is recovered from the selected cells. In other preferred embodiments, the cells are bacteria or yeast. Other cells for use in the invention include mammalian cells. Preferred surface proteins include flagella proteins, receptors, and any other protein with an extracellular domain. In other preferred embodiments, one or more nucleic acids encoding a protein or all of the proteins required for the synthesis of the displayed small molecule or posttranslational modification are contained in the genome of the cell (e.g., in a plasmid, artificial chromosome, or endogenous chromosome in the cell). In still other preferred embodiments, the cell is propagated to generate additional cells that display the selected small molecule or posttranslational modification.

In preferred embodiments of any of the above selection methods, the small molecule or posttranslational modification is a biotin, biotin analog, lipid, phosphopantetheine group, carbohydrate, prosthetic group, vitamin, ketone, carboxylic acid, alkaloid, terpene, polyketide, or polypeptide. In some embodiments, the small molecule, posttranslational modification, or prosthetic group is not biotin. In particular embodiments, the lipid is covalently attached to a phosphopantetheinylated amino acid in the display peptide (e.g., an acyl carrier protein, acyl carrier protein domain, thiolation domain, or thioesterase domain). In other embodiments, the lipid is a palmitoyl group, myristoyl group, farnesyl group, geranylgeranyl group, lipoyl group, arachidonic acid, or steroid. Preferred carbohydrate modifications include the addition of a chondroitin sulfate, heparan sulfate, or keratan sulfate. A preferred prosthetic group is heme. Each virus or cell may display one or more copies of the same protein fusion or may display one or more copies of different protein fusions (such as 2, 3, 4, 5, or more different protein fusions). Preferably, one or more selected viruses or cells displays a novel small molecule or a novel posttranslational modification. In preferred embodiments, a virus or cell expressing different protein fusions expresses different small molecules or different posttranslational modifications.

In other embodiments of the above aspects, a nucleic acid in the cells is mutated prior to the expression of the protein fusion. The nucleic acid that is mutated may be an endogenous or a heterologous nucleic acid. The mutated nucleic acid may also be a duplicated copy of an endogenous nucleic acid. A preferred mutagenesis technique involves replacing a nucleic acid with a heterologous nucleic acid, such as a nucleic acid that has been modified by site-directed mutagenesis using the polymerase chain reaction (PCR) or error-prone PCR to contain a mutation. Alternatively, some or all of a nucleic acid sequence may be mutated by shuffling or other type of DNA rearrangement methods. Another mutagenesis method that may be exploited involves contacting the cells with a mutagenic agent. Preferably, the nucleic acid that is mutated encodes a biotin ligase, phosphopantetheinyl transferase, fatty acid synthase, polyketide synthase, nonribosomal peptide synthase, lipoate ligase, glycosyltransferase, farnesyltransferase, or geranylgeranyltransferase. The cell may also contain a naturally-occurring version of one or more heterologous nucleic acids. For example, the cell may be genetically modified to contain one or more heterologous polyketide synthase, nonribosomal peptide synthase, or fatty acid synthase nucleic acids.

In yet another preferred embodiment, the target molecule is immobilized. Useful solid supports for immobilizing target molecules include any rigid or semi-rigid surface that may be derivatized to react with the target molecule. The support can be any porous or non-porous water insoluble material, including, without limitation, membranes, filters, chips, magnetic or nonmagnetic beads, and polymers. Preferred target molecules may include a detectable label or bind an affinity reagent. In another preferred embodiment, the target molecule is fluorescent, and the viruses or cells are sorted based on fluorescence intensity after they are contacted with the target molecule. Exemplary target compounds that may be used in this method include organic molecules having a molecular weight less than 1000, 500, or 250 daltons; proteins (e.g., antibodies, virulence factors, cytokines, hormones, ligands, or receptors); lipids; carbohydrates; nucleic acids; and infectious agents (e.g., viruses, bacteria, parasites, fungi, protozoa, or other eukaryotic pathogens). In various embodiments, the target protein contains a purification tag, such as a hexahistidine, maltose-binding protein, FLAG, or myc tag.

The invention also provides viruses and cells that express a small molecule or posttranslational modification on their surface. These viruses and cells are useful for the selection of displayed compounds that bind target molecules of interest. According to this aspect of the invention, a virus is provided that expresses on its surface a protein fusion which includes a surface protein covalently linked to a display peptide. In various embodiments, the display peptide is modified by a biotin analog, phosphopantetheine, prosthetic group other than biotin, ketone, terpene, alkaloid, polyketide, palmitoyl group, myristoyl group, farnesyl group, geranylgeranyl group, lipoyl group, arachidonic acid, steroid, chondroitin sulfate, heparan sulfate, keratan sulfate, or a molecule including an unnatural amino acid. In some embodiments, the display peptide is modified by a small molecule that (i) is covalently linked to a side-chain of an amino acid in the display peptide, (ii) has an unnatural amino acid, (iii) has a molecular weight less than 4,000 daltons and has either an unnatural amino acid or a moiety other than an amino acid, or (iv) has a molecular weight less than 2,000 daltons. In some embodiments, the small molecule is not biotin. Preferably, the small molecule binds a target molecule of interest.

In a related aspect, the invention provides a virus that expresses on its surface a protein fusion that includes a surface protein covalently linked to a posttranslationally modified display peptide. In various embodiments, the display peptide is modified by a biotin analog, phosphopantetheine, prosthetic group other than biotin, ketone, terpene, alkaloid, polyketide, palmitoyl group, myristoyl group, farnesyl group, geranylgeranyl group, lipoyl group, arachidonic acid, steroid, chondroitin sulfate, heparan sulfate, keratan sulfate, or a molecule including an unnatural amino acid. In some embodiments, the posttranslational modification is not biotin. Preferably, the posttranslational modification attached to the display peptide binds a target molecule.

In another related aspect, the invention provides a virus expressing on its surface a protein fusion comprising a surface protein covalently linked to a display peptide. A lipid, polyketide, or polypeptide is covalently bound to a phosphopantetheinylated amino acid in the display peptide. Preferably, the display peptide is an acyl carrier protein, acyl carrier protein domain, thiolation domain, or thioesterase domain.

Preferred viruses of any of the above aspects include filamentous and non-filamentous bacteriophage (such as M13, fl, and fd). The viruses may be used to infect a variety of bacteria, such as Escherichia (e.g., E. coli) or Salmonella. Preferably, the surface protein is a viral coat protein (e.g, pIII or pVIII). In other preferred embodiments, the displayed small molecule or posttranslational modification is a biotin, biotin analog, lipid, phosphopantetheine group, carbohydrate, prosthetic group, vitamin, ketone, carboxylic acid, alkaloid, terpene, polyketide, or polypeptide. In some embodiments, the small molecule, posttranslational modification, or prosthetic group is not biotin. In particular embodiments, the lipid is covalently attached to a phosphopantetheinylated amino acid in the display peptide (e.g., an acyl carrier protein, acyl carrier protein domain, thiolation domain, or thioesterase domain). In other embodiments, the lipid is a palmitoyl group, myristoyl group, farnesyl group, geranylgeranyl group, lipoyl group, arachidonic acid, or steroid. Preferred carbohydrates include chondroitin sulfate, heparan sulfate, and keratan sulfate. A preferred prosthetic group is heme. Preferably, the virus displays a novel small molecule or a novel posttranslational modification.

In other preferred embodiments of any of the above aspects related to viruses, one or more nucleic acids of the virus encodes a protein required for the synthesis of the small molecule, posttranslational modification, lipid, polyketide, or polypeptide. In particular embodiments, the nucleic acid encodes a biotin ligase, phosphopantetheinyl transferase, fatty acid synthase, polyketide synthase, nonribosomal peptide synthase, lipoate ligase, glycosyltransferase, farnesyltransferase, or geranylgeranyltransferase. Preferably, the nucleic acid has a mutation.

The invention also provides cells expressing small molecules or posttranslational modifications which preferably bind a target molecule. In one such aspect, a cell is provided that expresses on its surface a protein fusion which includes a surface protein covalently linked to a display peptide. In various embodiments, the display peptide is modified by a biotin analog, phosphopantetheine, prosthetic group other than biotin, ketone, terpene, alkaloid, polyketide, palmitoyl group, myristoyl group, farnesyl group, geranylgeranyl group, lipoyl group, arachidonic acid, steroid, chondroitin sulfate, heparan sulfate, keratan sulfate, or a molecule including an unnatural amino acid. In some embodiments, the display peptide is modified by a small molecule that (i) is covalently attached to a side-chain of an amino acid in the display peptide, (ii) has an unnatural amino acid, (iii) has a molecular weight less than 4,000 daltons and has either an unnatural amino acid or a moiety other than an amino acid, or (iv) has a molecular weight less than 2,000 daltons. In some embodiments, the small molecule is not biotin. Preferably, the small molecule binds a target molecule of interest.

In a related aspect, the invention provides a cell that expresses on its surface a protein fusion that includes a surface protein covalently linked to a posttranslationally modified display peptide. In various embodiments, the display peptide is modified by a biotin analog, phosphopantetheine, prosthetic group other than biotin, ketone, terpene, alkaloid, polyketide, palmitoyl group, myristoyl group, farnesyl group, geranylgeranyl group, lipoyl group, arachidonic acid, steroid, chondroitin sulfate, heparan sulfate, keratan sulfate, or a molecule including an unnatural amino acid. In some embodiments, the posttranslational modification is not biotin. Preferably, the posttranslational modification attached to the display peptide binds a target molecule.

In another related aspect, the invention provides a cell expressing on its surface a protein fusion comprising a surface protein covalently linked to a display peptide. A lipid, polyketide, or polypeptide is covalently bound to a phosphopantetheinylated amino acid in the display peptide. Preferably, the display peptide is an acyl carrier protein, acyl carrier protein domain, thiolation domain, or thioesterase domain.

Preferred cells of any of the above aspects include bacteria (e.g., E. coli, Bacillus subtilis) and yeast (e.g., S. cerevisiae). Other cells for use in the invention include mammalian cells. Preferred surface proteins include flagella proteins, receptors, and any other protein with an extracellular domain. Preferably, the displayed small molecule or posttranslational modification is a biotin, biotin analog, lipid, phosphopantetheine group, carbohydrate, prosthetic group, vitamin, ketone, carboxylic acid, alkaloid, terpene, polyketide, or polypeptide. In some embodiments, the small molecule, posttranslational modification, or prosthetic group is not biotin. In particular embodiments, the lipid is covalently attached to a phosphopantetheinylated amino acid in the display peptide (e.g., an acyl carrier protein, acyl carrier protein domain, thioesterase domain, or thiolation domain). In other embodiments, the lipid is a palmitoyl group, myristoyl group, farnesyl group, geranylgeranyl group, lipoyl group, arachidonic acid, or steroid. Preferred carbohydrates include chondroitin sulfate, heparan sulfate, and keratan sulfate. A preferred prosthetic group is heme. Preferably, the cell displays a novel small molecule or a novel posttranslational modification. In other preferred embodiments, one or more nucleic acids of the cell encodes a protein required for the synthesis of the small molecule, posttranslational modification, lipid, polyketide, or polypeptide.

Other preferred cells of any of the above aspects contains one or more nucleic acids with spontaneous or artificially induced mutations. A preferred mutagenesis technique involves replacing a nucleic acid with a heterologous nucleic acid, such as a nucleic acid that has been modified by site-directed mutagenesis using PCR or error-prone PCR to contain a mutation. Alternatively, some or all of a nucleic acid sequence may be mutated by shuffling or other type of DNA rearrangement methods. Another mutagenesis method that may be exploited involves contacting the cells with a mutagenic agent. The nucleic acid that is mutated may be an endogenous or a heterologous nucleic acid. The mutated nucleic acid may also be a duplicated copy of an endogenous nucleic acid. In other preferred embodiments, one or more mutated or heterologous nucleic acids encodes a protein required for the synthesis of the small molecule or posttranslational modification. Preferably, the nucleic acid that is mutated encodes a biotin ligase, phosphopantetheinyl transferase, fatty acid synthase, polyketide synthase, nonribosomal peptide synthase, lipoate ligase, glycosyltransferase, farnesyltransferase, or geranylgeranyltransferase. The cell may also contain a naturally-occurring version of one or more heterologous nucleic acids. For example, the cell may be genetically modified to contain one or more heterologous polyketide synthase, nonribosomal peptide synthase, or fatty acid synthase nucleic acids.

Additionally, the invention provides protein fusions that include a surface protein covalently linked to a display peptide for a modification, such as the addition of a novel small molecule or a novel posttranslational modification. The protein fusions and the nucleic acids encoding them may be used to express novel or naturally-occurring molecules on the surface of viruses or cells.

In one such aspect, the invention features a protein fusion that includes a surface protein covalently linked to a display peptide capable of being modified with a small molecule. In various embodiments, the display peptide is modified by a biotin analog, phosphopantetheine, prosthetic group other than biotin, ketone, terpene, alkaloid, polyketide, palmitoyl group, myristoyl group, farnesyl group, geranylgeranyl group, lipoyl group, arachidonic acid, steroid, chondroitin sulfate, heparan sulfate, keratan sulfate, or a molecule including an unnatural amino acid. In some embodiments, the small molecule (i) is covalently attached to a side-chain of an amino acid in the display peptide, (ii) has an unnatural amino acid, (iii) has a molecular weight less than 4,000 daltons and has either an unnatural amino acid or a moiety other than an amino acid, or (iv) has a molecular weight less than 2,000 daltons. In some embodiments, the small molecule is not biotin. Preferably, the small molecule binds a target molecule of interest.

In a related aspect, the invention provides a protein fusion that includes a surface protein covalently linked to a posttranslationally modified display peptide. In preferred embodiments, the display peptide is modified by a biotin analog, phosphopantetheine, prosthetic group other than biotin, ketone, terpene, alkaloid, polyketide, palmitoyl group, myristoyl group, farnesyl group, geranylgeranyl group, lipoyl group, arachidonic acid, steroid, chondroitin sulfate, heparan sulfate, keratan sulfate, or a molecule including an unnatural amino acid. In some embodiments, the posttranslational modification is not biotin. Preferably, the posttranslational modification attached to the display peptide binds a target molecule.

Preferred protein fusions of any of the above aspects include a flagella protein, cell receptor, or viral coat protein as the surface protein component. Preferably, the small molecule or posttranslational modifications is a biotin, biotin analog, lipid, phosphopantetheine group, carbohydrate, prosthetic group, vitamin, ketone, carboxylic acid, alkaloid, terpene, polyketide, or polypeptide. In some embodiments, the small molecule, posttranslational modification, or prosthetic group is not biotin. In particular embodiments, the lipid is covalently attached to a phosphopantetheinylated amino acid in the display peptide (e.g., an acyl carrier protein). In other embodiments, the lipid is a palmitoyl group, myristoyl group, farnesyl group, geranylgeranyl group, lipoyl group, arachidonic acid, or steroid. Preferred carbohydrates include chondroitin sulfate, heparan sulfate, and keratan sulfate. A preferred prosthetic group is heme. Preferably, the protein fusion displays a novel small molecule or a novel posttranslational modification.

In a related aspect, the invention provides a nucleic acid which encodes a protein fusion of the invention. Preferably, the nucleic acid is contained in a vector and operably linked to a promoter. The promoter may be a heterologous promoter or a promoter that is naturally associated with the surface protein that is part of the protein fusion.

In various embodiments of any of the above aspects, the bacteria are Escherichia (e.g., E. coli), Salmonella (e.g., Salmonella typhimurium), Shigella (e.g., Shigella sonnei), or Bacillus (e.g, Bacillus subtilis). In some embodiments, the bacteria are bacterial spores, such as Bacillus subtilis spores. Preferred yeast include Saccharomyces cerevisiae. Preferred small molecules or posttranslational modifications include cyclic compounds, such as cyclic polyketides or nonribosomally synthesized polypeptides. In other preferred embodiments, the display peptide or the protein fusion is not phosphorylated.

In various embodiments of any of the aspects of the invention, the small molecule or posttranslational modification includes one or more alkyl groups, such as a linear or branched saturated hydrocarbon group of 1-5, 1-10, 1-20, 1-50, or 1-100 carbon atoms. Exemplary alkyl groups include methyl, ethyl, n-propyl, isopropyl, n-butyl, isobutyl, t-butyl, octyl, decyl, and tetradecyl groups; and cycloalkyl groups, such as cyclopentyl and cyclohexyl groups.

In other embodiments, the small molecule or posttranslational modification has one or more alkenyl groups, such as a linear or branched hydrocarbon group of 1-5, 1-10, 1-20, 1-50, or 1-100 carbon atoms containing at least one carbon-carbon double bond. In still other embodiments, the small molecule or posttranslational modification has one or more alkynyl groups, such as a linear or branched hydrocarbon group of 1-5, 1-10, 1-20, 1-50, or 1-100 carbon atoms containing at least one carbon-carbon triple bond.

Other exemplary groups the may be present in a small molecule or posttranslational modification include heteroalkyl, heteroalkenyl, and heteroalkynyl groups in which one or more carbons from an alkyl, alkenyl, or alkynyl group have been replaced with another atom, such as nitrogen, sulfur, oxygen, or phosphate. One or more of the hydrogens in an alkyl, alkenyl, or alkynyl group may be optionally substituted with a hydroxy, cyano, thio, halo (e.g., chloro, fluoro, iodo, or bromo), nitro, amino, aryl, alkoxy, or acyl group.

In still other embodiments, the small molecule or posttranslational modification has an aryl group, such as a monovalent aromatic hydrocarbon radical consisting of one or more rings in which at least one ring is aromatic in nature, which may optionally be substituted with one of the following substituents: hydroxy, cyano, alkyl, alkoxy, thioalkyl, halo, haloalkyl, hydroxyalkyl, nitro, amino, alkylamino, diakylamino, or acyl. Other suitable groups include heteroaryl groups in which one or more carbons in a ring have been replaced with another atom, such as nitrogen, sulfur, or oxygen. Yet other suitable aryl groups contain one or more nitro, halo, aryl, alkyl, alkoxy, or acyl substituents.

In yet other embodiments, the small molecule or posttranslational modification has one or more alkoxy or acyl groups. Preferred alkoxy groups have the formula —OR, and exemplary acyl groups have the formula —C(O)R, wherein R is an alkyl or aryl group as defined above. Examples of alkoxy groups include, but are not limited to, methoxy, ethoxy, and isopropoxy groups. Examples of acyl groups include acetyl and benzoyl groups.

Examples of carbohydrate groups that may be included in a small molecule or posttranslational modification are monosaccharides that have an aldehyde group (i.e., aldoses) or a keto group (i.e., ketoses), disaccharides, and other oligosaccharides. Carbohydrates may be linear or cyclic, and they may exist in a variety of conformations. Other carbohydrates include those that have been modified (e.g., wherein one or more of the hydroxyl groups are replaced with halogen, alkoxy moieties, aliphatic groups, or are functionalized as ethers, esters, amines, or carboxylic acids). Examples of modified carbohydrates include α- or β-glycosides such as methyl α-D-glucopyranoside or methyl β-D-glucopyranoside; N-glycosylamines; N-glycosides; D-gluconic acid; D-glucosamine; D-galactosamine; and N-acteyl-D-glucosamine.

It is also contemplated that the surface protein component of the protein fusion may be modified instead of, or in addition to, the modification of the display peptide component of the protein fusion. For example, a surface protein that includes all or part of a cell receptor may be glycosylated.

As used herein, the term “protein” includes any two or more amino acids, or amino acid analogs or derivatives, joined by peptide bond(s), regardless of length or posttranslational modification. This term includes proteins, peptides, and polypeptides.

By “surface protein” is meant any viral coat protein or any protein that contains one or more extracellullar domains. The extracellular domains of a surface protein may be expressed, for example, on the external surface of the cytoplasmic membrane of gram positive bacteria, the outer membrane of gram negative bacteria, the cell wall of yeast, or the plasma membrane of mammalian cells. Preferred surface proteins include transmembrane proteins. Other preferred surface proteins include flagella protein (e.g., FliC), receptors, and protein involved in cell adhesion (e.g., Aga2p). Preferred viral coat proteins include pIII and pVIII. Still other preferred surface proteins include proteins that have a sequence at least 50, 60, 70, 80, 90, 95, or 100% identical to the sequence of a naturally-occurring endogenous or heterologous surface protein. Other suitable surface proteins are proteins having a region of consecutive amino acids that is identical to the corresponding region of a preferred surface protein (e.g., a region of at least 25, 50, 100, 200, or 500 amino acids) but is less than the full-length sequence.

By “display peptide” is meant a peptide capable of being modified and expressed on the surface of a virus or cell. Display peptides can contain any number of amino acids. For example, display peptides may contain as few as 50, 40, 30, 20, or less residues or as many as 100, 150, 200 or more residues.

By “covalently linked” is meant covalently bonded or connected through a series of covalent bonds. For example, a surface protein may be directly bonded to a display peptide or connected to the display peptide through a linker (e.g., a linker of at least 5, 10, 20, or 50 amino acids).

By “small molecule” is meant an organic compound or a moiety from an organic compound that can modify a protein fusion of the present invention. Typically, the small molecule is covalently attached to the display peptide component of a protein fusion. Preferred small molecules include compounds or moieties that are covalently linked to a side-chain of an amino acid in a display peptide. Examples of amino acid side-chains that may be modified include the side chains of a serine, threonine, cysteine, methionine, tyrosine, tryptophan, histidine, aspartic acid, glutamic acid, aparagine, glutamine, or lysine residue. Other preferred small molecules have 1, 2, 3, 4, 5, 6, 8, 10, or more unnatural amino acids. Still other preferred small molecules have a moiety other than an amino acid. For example, in some embodiments, the small molecule does not consist entirely of amino acids or is not a peptide. Preferably, the small molecule has a molecular weight less than 10,000, 8,000, 6,000, 5,000, 4,000, 3,500, 3,000, 2,500, 2,000, 1,500, 1,000, 750, 500, 400, 300, 250, 200, or 100 daltons. In still other preferred embodiments, the small molecule has a molecular weight contained in one of the following ranges: 100-4,000 daltons, 100-3,000 daltons; 100-2,000 daltons; 100-1,000 daltons; 100-750 daltons; 250-4,000 daltons, 250-3,000 daltons; 250-2,000 daltons; 250-1,000 daltons; 250-750 daltons; 400-4,000 daltons, 400-3,000 daltons; 400-2,000 daltons; 400-1,000 daltons; or 400-750 daltons, inclusive. More preferably, the molecular weight of the small molecule is between 250-2,000 daltons. The small molecule may be attached to the display peptide either during the translation of the display peptide, after the translation of the display peptide, or after the translation of the entire protein fusion. The small molecule may be a naturally-occurring or non-naturally-occurring compound.

By “posttranslational modification” is meant an organic compound or a moiety from an organic compound that can modify a protein fusion of the present invention after the translation of the display peptide or, more preferably, after the translation of the entire protein fusion. A posttranslational modification does not include a naturally-occurring L-amino acid that is added to the amino group of the amino-terminus or added to the carboxylic acid of the carboxy-terminus of the display peptide or the protein fusion during the translation of the display peptide or protein fusion.

By “unnatural amino acid” is meant an amino acid or amino acid analog other than any of the 20 naturally-occurring L-amino acids that are found in proteins. For example, the unnatural amino acid may be the D-isomer of a naturally-occurring L-amino acid. Other exemplary unnatural amino acids include nonproteinogenic residues or amino acid analogs, such as β-amino acids (e.g., β-alanine), hydroxy acids, N-methylated acids, cyclohexylalanine, ethylglycine, norleucine, norvaline, allo-isoleucine, homocysteine, homoserine, homophenylalanine, and 3-aminobutyric acid (von Dohren et al., Chem. Biol. 10:R273-279, 1999).

By “biotin ligase” is meant one or more enzymes that catalyze the covalent attachment of biotin or a biotin analog to another protein or peptide (e.g., a display peptide component of a protein fusion of the invention). Preferred biotin ligases include E. coli BirA and proteins that have a region of consecutive amino acids that is substantially identical to the corresponding region of BirA. Preferably, this region of BirA includes at least 60, 70, 80, 90, 95, or 100% of the amino acids of BirA.

By “phosphopantetheinyl transferase” is meant one or more enzymes that catalyze the covalent attachment of 4′-phosphopantetheine or an analog thereof to another protein or peptide (e.g., a display peptide component of a protein fusion of the invention). Preferred phosphopantetheinyl transferases include ACP-synthases which catalyze the attachment of 4′-phosphopantetheine to an acyl carrier protein (ACP) or to an ACP-domain of a multidomain enzyme, such as a polyketide synthase, a nonribosomal peptide synthase, or a hybrid polyketide/nonribosomal peptide synthase. Other preferred phosphopantetheinyl transferases include enzymes which catalyze the attachment of 4′-phosphopantetheine to an peptidyl carrier protein-domain (PCP) of a multidomain enzyme, such as a polyketide synthase, a nonribosomal peptide synthase, or a hybrid polyketide/nonribosomal peptide synthase. Still other preferred phosphopantetheinyl transferases include proteins that have a region of consecutive amino acids that is substantially identical to the corresponding region of E. coli ACP-synthase. Preferably, this region of E. coli ACP-synthase includes at least 60, 70, 80, 90, 95, or 100% of the amino acids of E. coli ACP-synthase.

By “acyl carrier protein (ACP) or ACP-domain” is meant a protein or a domain of a multidomain protein that may be modified by the covalent attachment of 4′-phosphopantetheine or an analog of 4′-phosphopantetheine. During fatty acid synthesis, the free thiol group of the 4′-phosphopantetheine is modified by the attachment of a fatty acid or a component of a fatty acid. During polyketide synthesis, the free thiol group of the 4′-phosphopantetheine is modified by the attachment of an acyl group, such as an acyl group containing a two or three carbon moiety derived from coenzyme A (CoA) or a CoA derivative (O'Hagan, The polyketide metabolites, Ellis Horwood (ed), Chichester, U.K., 1991). For example, the acyl group may be derived from propionyl-CoA or methylmalonyl CoA. Preferred ACPs include E. coli ACP, nodulation protein (nodF) from Rhizobium meliloti (accession number A24706), nodulation protein (nod F) from Rhizobium leguminosarum (accession number CAA27355.1), nodF protein from Mesorhizobium loti (accession number AP003005), acyl carrier protein from Cuphea lanceolata (accession numbers X77621 and S42026), acyl carrier protein I precursor from Spinacia oleracea (accession numbers M17636 and 1410328A), acyl carrier protein II from Spinacia oleracea (accession number X52065), acyl carrier protein from Coriandrum sativum (accession number AF083950), acyl carrier protein from Capsicum chinense (accession number AF127796), acyl carrier protein from Casuarina glauca (accession number Y10994), and acyl carrier protein from Fragaria vesca (accession number AJ001446).

By “peptidyl carrier protein domain (PCP)” is meant a domain of a multidomain protein that may be modified by the covalent attachment of 4′-phosphopantetheine or an analog of 4′-phosphopantetheine. The free thiol group of the 4′-phosphopantetheine is typically modified by the attachment of an amino acid or amino acid analog.

By “fatty acid synthase” is meant one or more enzymes that catalyze one or more reactions required for the formation of a fatty acid. For example, a fatty acid synthase may transfer an acyl group to a phosphopantetheinylated ACP or ACP-domain. Preferred fatty acid synthases include E. coli fatty acid synthase, conidial green pigment synthase (accession number Q03149), putative polyketide or fatty acid synthase from Aspergillus nidulans (accession number X65866), and protein MxaC from Stigmatella aurantiaca (accession number AF319998). Other exemplary fatty acid synthases have a region of consecutive amino acids that is substantially identical to the corresponding region of a preferred fatty acid synthase. Preferably, this region of substantial identity includes at least 60, 70, 80, 90, 95, or 100% of the amino acids of a preferred fatty acid synthase. Given the high degree of homology between fatty acid synthases, polyketide synthases, and noribosomal peptide synthases, it is also contemplated that a polyketide synthase or noribosomal peptide synthase, such as those described herein, may be used as a fatty acid synthase. For example, Metz et al. have reported the production of polyunsaturated fatty acids by polyketide synthases in both prokaryotes and eukaryotes (Science 293:290-293, 2001).

By “polyketide synthase” is meant one or more enzymes that catalyze a reaction required for the formation of polyketide. Polyketides comprise a diverse group of natural products synthesized via linear repetitive condensation of β-ketones. For example, a polyketide synthase may catalyze the covalent attachment of a new functional group (e.g., an acyl or substituted acyl group), to an intermediate in the synthesis of a polyketide. Preferred polyketide synthases include type I polyketide synthase from Exophiala dermatitidis (accession number AF130309), conidial green pigment synthase (accession number Q03149), probable polyketide synthase from Emericella nidulans (accession number S28353), putative polyketide or fatty acid synthase from Aspergillus nidulans (accession number X65866), polyketide synthase from Aspergillus parasiticus (accession number L42766), polyketide synthase from Gibberella fujikuroi (accession number AJ278141), polyketide synthase from Aspergillus fumigatus (accession number AF025541), polyketide synthase from Nodulisporium sp. ATCC74245 (accession number AF151533), polyketide synthase from Colletotrichum lagenarium (accession number D83643), and protein MxaC from Stigmatella aurantiaca (accession number AF319998). Other exemplary polyketide synthases have a region of consecutive amino acids that is substantially identical to the corresponding region of a preferred polyketide synthase. Preferably, this region of substantial identity includes at least 60, 70, 80, 90, 95, or 100% of the amino acids of a preferred polyketide synthase. Given the high degree of homology between polyketide synthases, noribosomal peptide synthases, and fatty acid synthases, it is also contemplated that a noribosomal peptide synthase or fatty acid synthase, such as those described herein, may be used as a polyketide synthase.

By “nonribosomal peptide synthase” is meant one or more enzymes that catalyze one or more reactions required for the formation of a nonribosomally synthesized polypeptide. For example, a nonribosomal peptide synthase may catalyze the covalent attachment of an amino acid or amino acid analog to an intermediate in the synthesis of a nonribosomally synthesized peptide. Preferred nonribosomal peptide synthases include tyrocidine synthases, bacterial and fungal nonribosomal peptide synthases, and proteins that have a region of consecutive amino acids that is substantially identical to the corresponding region of a bacterial nonribosomal peptide synthase. Preferably, this region of the bacterial nonribosomal peptide synthase includes at least 60, 70, 80, 90, 95, or 100% of the amino acids of the bacterial nonribosomal peptide synthase. Given the high degree of homology between noribosomal peptide synthases, polyketide synthases, and fatty acid synthases, it is also contemplated that a polyketide synthase or fatty acid synthase, such as those described herein, may be used as a noribosomal peptide synthase.

By “hybrid polyketide/nonribosomal peptide synthase” is meant one or more synthases that have a domain typically found in a polyketide synthase and a domain typically found in a nonribosomal peptide synthase. For example, a hybrid polyketide/nonribosomal peptide synthase may catalyze the covalent attachment of an amino acid and a small molecule to an intermediate in the synthesis of a polyketide. Preferred hybrid polyketide/nonribosomal peptide synthases include bacterial and/or fungal hybrid polyketide/nonribosomal peptide synthases and proteins that have a region of consecutive amino acids that is substantially identical to the corresponding region of a bacterial hybrid polyketide/nonribosomal peptide synthase. Preferably, this region of the hybrid polyketide/nonribosomal peptide synthase includes at least 60, 70, 80, 90, 95, or 100% of the amino acids of the bacterial hybrid polyketide/nonribosomal peptide synthase.

By “lipoate ligase” is meant one or more enzymes that catalyze the covalent attachment of lipoate or a lipoate analog to another protein or peptide (e.g., a display peptide component of a protein fusion of the invention). Preferred lipoate ligases include E. coli LplA and proteins that have a region of consecutive amino acids that is substantially identical to the corresponding region of E. coli LplA. Preferably, this region of E. coli LplA includes at least 60, 70, 80, 90, 95, or 100% of the amino acids of E. coli LplA.

By “glycosyltransferase” is meant one or more enzymes that catalyze the covalent transfer of a carbohydrate to another protein or peptide (e.g., a display peptide component of a protein fusion of the invention). Preferred glycosyltransferases include yeast glycosyltransferases and proteins that have a region of consecutive amino acids that is substantially identical to the corresponding region of a yeast glycosyltransferase. Preferably, this region of the yeast glycosyltransferase includes at least 60, 70, 80, 90, 95, or 100% of the amino acids of the yeast glycosyltransferase.

By “farnesyltransferase” is meant one or more enzymes that catalyze the covalent transfer of a farnesyl group or an analog thereof to another protein or peptide (e.g., a display peptide component of a protein fusion of the invention). Preferred farnesyltransferases include yeast farnesyltransferases and proteins that have a region of consecutive amino acids that is substantially identical to the corresponding region of a yeast farnesyltransferase. Preferably, this region of the yeast farnesyltransferase includes at least 60, 70, 80, 90, 95, or 100% of the amino acids of the yeast farnesyltransferase.

By “geranylgeranyltransferase” is meant one or more enzymes that catalyze the covalent attachment of a geranylgeranyl group or an analog thereof to another protein or peptide (e.g., a display peptide component of a protein fusion of the invention). Preferred geranylgeranyltransferases include yeast geranylgeranyltransferases and proteins that have a region of consecutive amino acids that is substantially identical to the corresponding region of a yeast geranylgeranyltransferase. Preferably, this region of the yeast geranylgeranyltransferase includes at least 60, 70, 80, 90, 95, or 100% of the amino acids of the yeast geranylgeranyltransferase.

By a “detectable label” is meant any means for marking or detecting the presence of a molecule. Detectable labels are well known in the art and include, without limitation, radioactive labels (e.g., isotopes such as ³²P or ³⁵S) and nonradioactive labels (e.g., chemiluminescent labels or fluorescent labels, e.g., fluorescein). The label used may itself be detectable (e.g., radioisotope labels or fluorescent labels) or, in the case of an enzymatic label, may catalyze chemical alteration of a support compound or composition which is detectable.

By an “affinity reagent” is meant any molecule that specifically binds (e.g., has an affinity Kₐ>10⁴ M⁻¹), covalently or non-covalently, to another molecule. Affinity reagents include nucleic acids, proteins, and compounds (such as small molecules), and include members of antibody-antigen (or hapten) pairs, ligand-receptor pairs, biotin-avidin pairs, polynucleotides with complementary base pairs (for example, oligonucleotide tags), and the like.

By “population of viruses or cells” is meant more than one virus or cell. The populations of viruses or cells may express any number of different small molecules or posttranslational modifications. For example, the population may express as few as 10, 10², 10⁹, or 10¹¹ different molecules or as many as 10¹³, 10¹⁴, 10¹⁵ or more different molecules.

By “selecting” is meant substantially partitioning a virus or cell from other viruses or cells in a population. Preferably, the partitioning provides at least a 2-fold, preferably, a 30-fold, more preferably, a 100-fold, and most preferably, a 1,000-fold enrichment of a desired molecule relative to undesired molecules in a population following the selection step. The selection step may be repeated a number of times, and different types of selection steps may be combined in a given approach. The population preferably contains at least 10⁹ viruses or cells, more preferably at least 10¹¹, 10¹³, or 10¹⁴ viruses or cells and, most preferably, at least 10¹⁵ viruses or cells.

By “recovered from” is meant substantially isolating (that is, at least a 2-fold purification) or identifying a moiety that is part of a small molecule or posttranslational modification expressed by a selected virus or cell. A small molecule or posttranslational modification that remains on a display peptide may be characterized by standard techniques such as mass spectrometry or NMR. Alternatively, a compound containing all, or part of, the small molecule or posttranslational modification may be cleaved from a modified display peptide and then characterized. If desired, the compound may be further purified using standard methods such as extraction, precipitation, column chromatography, magnetic bead purification, and panning with a plate-bound target molecule.

By “mutation” is meant an alteration in a naturally-occurring or reference nucleic acid sequence, such as an insertion, deletion, inversion, or nucleotide substitution. Preferably, the amino acid sequence encoded by the nucleic acid sequence has at least one amino acid alteration from a naturally-occurring sequence. Examples of recombinant DNA techniques for altering the genomic sequence of a cell include inserting a DNA sequence from another organism (e.g., another bacteria, yeast, or mammalian genus or species) into the genome, deleting one or more DNA sequences, rearranging or shuffling DNA sequences, and introducing one or more base mutations (e.g., site-directed or random mutations) into a target DNA sequence.

By “substantially identical” is meant having a sequence that is at least 60, 70, 80, 90, or 100% identical to that of another sequence. Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications.

The present invention provides a number of advantages related to the generation, selection, and identification of compounds (e.g., small molecules) that bind target molecules of interest. In contrast to current methods that typically generate and select for relatively large nucleic acids and unmodified peptides, the present methods may be used to generate a variety of small candidate compounds (e.g., linear or cyclic small molecules). The present methods differ significantly from traditional display techniques because the present methods generate diversity through small molecules which are covalently linked to the protein fusion. The viruses and cells preferably contain within themselves, either in their genome or in artificial DNA inserts (e.g. plasmids, cosmids, or yeast artificial chromosomes), nucleic acids that encode the proteins responsible for the production of the molecules displayed on their surface. In this case, the selection of a small molecule also yields the genetic information that encodes its design. The present methods enable the display of nonribosomally synthesized small molecules on the surface of viruses and cells. For example, these small molecules include those produced by fatty acid synthases, nonribosomal peptide synthases, polyketide synthases, or other synthesis methods that do not originate from ribosomal synthesis. Differences between the display of ribosomal products using cell or viral display and some of the present nonribosomal display methods are illustrated in FIG. 9. In the present methods, the displayed molecule branches out from a display peptide. This enables the display of a wide variety of molecules of nonribosomal origin that can not be displayed using traditional approaches. Thus, the present methods greatly increase the diversity of candidate compounds that may be generated, displayed, and selected based on their affinity for a target molecule. This ability to generate a diverse set of small candidate compounds is important because most drug products are compounds with molecular weights less than 2,000 daltons or even smaller compounds with molecular weights less than 1,000 daltons.

The present methods are also advantageous in the speed with which large numbers of novel compounds may be generated, displayed, and selected. Because these novel compounds are displayed on the surfaces of viruses or cells, the compounds do not have to be isolated from intracellular compartments prior to testing for their ability to bind target molecules. Performing multiple rounds of selection enriches the population of candidate compounds for tight binders, all without the need for cell disruption. Furthermore, conducting multiple rounds of selection is typically less costly and more rapid than medicinal chemistry techniques for increasing affinities of potential drug molecules for their targets from, for example, the micromolar range to the nanomolar range. The present methods also provide a theoretically unlimited supply of the selected compounds because the selected cells may be easily cultured on a large scale (such as in a fermentor) to produce large quantities of the selected small molecules. In addition, these methods may be performed sequentially or simultaneously to select candidate compounds that bind a variety of target molecules.

Other features and advantages of the invention will be apparent from the following detailed description and from the claims.

### BRIEF DESCRIPTION OF THE DRAWINGS

FIG. 1A is the polynucleotide sequence and the encoded amino acid sequence for a display peptide that is biotinylated by the BirA biotin ligase. FIG. 1B is a schematic illustration of a vector, encoding an M13 bacteriophage containing this polynucleotide sequence linked to gene III, which encodes the bacteriophage pIII coat protein. FIG. 1C is a schematic illustration of a method for using this vector to transform an E. coli cell overexpressing BirA for the generation of bacteriophage expressing a biotinylated display peptide.

FIG. 2 is a schematic illustration of a fatty acid synthase (FAS, a fungal polyketide synthase (PKS), the lovastatin nonaketide synthase (NKS), and the lovastatin diketide synthase (LDKS) (adapted from Kennedy et al., Science 284:1368-1372). The arrangement of catalytic domains in fungal PKSs is the same as that for mammalian (rat) FASs. Fungal FASs and fungal PKSs have two subunits with a different order of catalytic domains. The following domains are illustrated: KS, β ketoacyl synthase; AT, acyltransferase; AT/MT, acetyl/malonyl transferase; DH, dehydratase; MeT, methyltransferase; ER, enoyl reductase [(ER), inactive ER]; KR, ketoreductase; ACP, acyl carrier protein; PT, product transfer; MT/PT, malonyl/palmityl transferase; and TE, thioesterase domains.

FIG. 3 is a schematic illustration of the protein template of the multiple carrier model in nonribosomal peptide biosynthesis (adapted from Mootz and Marahiel, Current Opin. in Chem. Biol. 1:543-551, 1997). As illustrated in the top of the figure, a module contains all the enzymatic activities required to incorporate a residue into the growing peptide chain. Within the modules (˜1100-1500 amino acids) a set of domains carries out single chemical reactions as outlined for the essential domains: adenylation (A, ˜550 amino acids), thiolation (T, ˜80 amino acids) and condensation (C, ˜450 amino acids). Other domains (e.g., epimerization and N-methylation domains) can chemically modify the incorporated residues.

FIG. 4 is a schematic illustration of chemical structures of exemplary peptide antibiotics and a siderophore produced by the nonribosomal pathway (adapted from Mootz and Marahiel, Current Opin. in Chem. Biol. 1:543-551, 1997). The cyclic decapeptide Tyrocidine A (D-Phe-Pro-Phe-D-Phe-Asn-Gln-Tyr-Val-omithine-Leu) is one of the prototypes synthesized on peptide synthase templates. The ergotamine (D-lysergic acid-Ala-Phe-Pro) is a precursor for ergot peptide alkaloids. Pristinamycin IA (3-hydroxypicolinic acid-Thr-aminobutyric acid-Pro-dimethylpara-aminophenylalanine-pipecolic acid-phenylglycine) is a good example of the structural variety of residues incorporated by peptide synthases. Enterobactin (dihydroxybenzoate-Ser) is an iron-chelating siderophore.

FIG. 5 is a schematic illustration of the biosynthesis of Tyrocidine A. Ten modules are responsible for the incorporation of each amino acid during the ordered synthesis of the linear decapeptide which is cyclized to generate the final product (adapted from Mootz et al., Proc. Natl. Acad. Sci. U.S.A. 97:5848-5853, 2000). Tyrocidine A (D-Phe-Pro-Phe-D-Phe-Asn-Gln-Tyr-Val-Om-Leu-)_{cyc} is produced by B. brevis ATCC 8185. Three peptide synthases TycA (124 kDa), TycB (405 kDa), and TycC (724 kDa), which are encoded by the genes tycA, tycB, and tycC, act in concert for the stepwise assembly of the cyclic decapeptide.

FIG. 6A is a schematic illustration of the reactions catalyzed by the D-Phe module and the L-Pro module of tyrocidine synthases. FIGS. 6B-6F are schematic illustrations of five strategies described in detail in Example 6 for expressing intermediates in the synthesis of Tyrocidine A on the surface of yeast, bacteriophage, or bacteria. Similar strategies can be applied to the display of any molecule of interest.

FIG. 7 is a schematic illustration of the 6-deoxyerythronolide B synthase (DEBS) which has the following catalytic domains: KS, ketosynthase; AT, acyl transferase; ACP, acyl carrier protein; KR, ketoreductase; ER, enoyl reductase; DH, dehydratase, and TE, thioesterase domains (adapted from Pfeifer et al., Science 291:1790-1792, 2001). DEBS utilizes 1 mole of propionyl-CoA and 6 moles of (2S)-methylmalonyl-CoA to synthesize 1 mole of 6-deoxyerythronolide B (6 dEB, compound 1).

FIG. 8 is a schematic illustration of a method for the generation of novel polyketides that are displayed on the surface of a bacteriophage. In this method, nucleic acids that encode modules from different polyketide synthases and/or nonribosomal peptide synthases are shuffled to generate different combinations of modules which produce polyketides containing different amino acids or amino acid analogs. These shuffled nucleic acids are used to transform E. coli for the production and display of polyketides on the surface of bacteriophage released from the bacteria.

FIG. 9 is a schematic illustration comparing traditional methods of bacteriophage/cell display to the present methods for displaying small molecules. As illustrated in this figure, traditional methods are used to display a ribosomally synthesized peptide or protein fused to either a viral coat protein or a cell-surface protein. In contrast, the present methods can be used to display a variety of small molecules of interest which are bound to a display peptide that is fused to either a coat protein or cell-surface protein and expressed on the surface of viruses or cells. In particular embodiments of the invention, nonribosomally synthesized small molecules are bound to an amino acid side chain in a display peptide (rather than to the amino or carboxy terminus of the display peptide) and thus branch out from the display peptide. In contrast, traditional display methods are limited to the generation of unmodified peptides or proteins attached through an amide bond to either the amino group of the N-terminal amino acid or the carboxyl group of the C-terminal amino acid of a viral coat protein or cell surface protein.

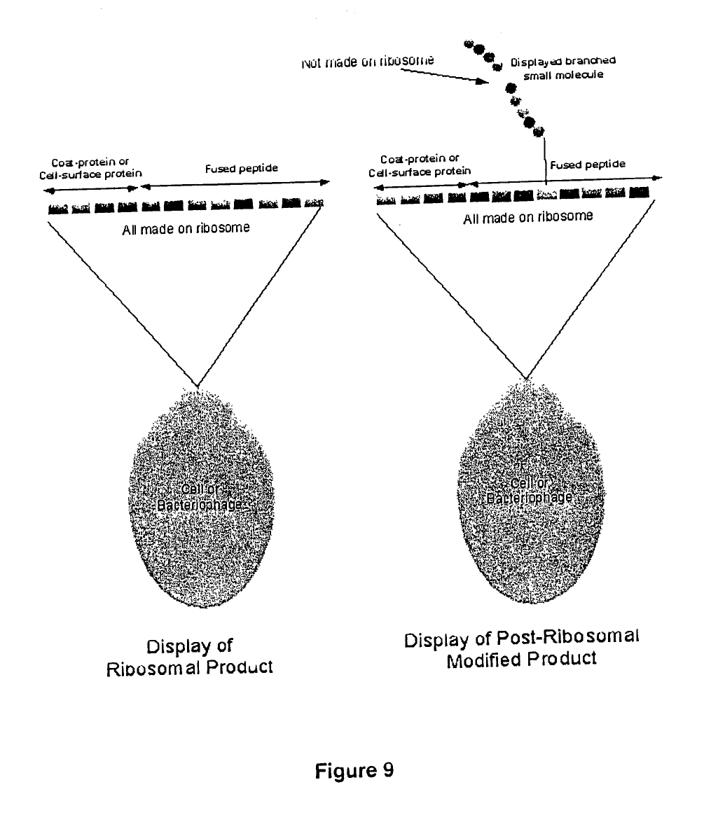

FIG. 10 is a non-denaturing polyacrylamide gel electrophoretic analysis of ACP. Lane 1 shows [2-¹⁴C] Malonyl-ACP, and lane 2 shows [³H] Acetyl-ACP.

### DETAILED DESCRIPTION

Novel methods have been developed to display a variety of organic molecules (e.g., small molecules) on the surface of viruses such as bacteriophage or on the surfaces of cells such as bacteria or yeast cells. In particular, the methods involve expressing protein fusions that contain (i) a display peptide that may be modified with an organic compound and (ii) a protein normally expressed on the surface of a virus or cell (e.g., a viral coat protein, flagella protein, cell receptor, or cell adhesion molecule). These protein fusions are expressed, and the display peptide components of the protein fusions are modified by organic molecules produced in the cells. For example, small molecules such as polyketide antibiotics, fatty acids, carbohydrates, steroids, alkaloids, or arachidonic acids may be attached to the display peptides. In some embodiments, the organic molecule is added after the translation of the display peptide as a posttranslational modification. The modified protein fusions are then transported to the surface of the bacteria, yeast, or mammalian cells.

To select the viruses or cells displaying compounds of interest (e.g., small molecules or posttranslational modifications which bind a target molecule), a population of viruses and/or cells displaying a wide variety of different molecules are contacted with a target molecule (for example, an immobilized target molecule, such as a target molecule bonded to magnetic beads). Preferably, each virus or cell displays one or more copies of a unique small molecule. Non-specific binding to the target molecule may be prevented by contacting the target molecule with underivatized viruses or cells prior to contacting the target molecule with the modified viruses or cells. The viruses or cells that bind with high affinity to the target molecule are preferentially captured and purified away from the vast majority of the viruses or cells. If desired, the selected viruses or cells may be re-cultured to produce a new population of viruses or cells enriched for high affinity binders. Cycles of binding and enrichment are carried out successively until the tight binders form the majority of the population (e.g., micromolar to nanomolar binders). In principle, a compound of interest in a population of 100 billion displayed compounds may be selected in this manner.

Essentially any molecule may be used as a target molecule to select compounds of interest. Exemplary target molecules include proteins with a known or unknown three-dimensional structure, membrane proteins stabilized in micelles, whole cells, or whole tissues. When both desirable and undesirable targets are available, cycles of selection and counter selection may be employed to improve the specificity of the compounds for their desired targets.

The molecular structures of the compounds of interest which bind the target molecule may be determined using standard methods. For example, modified displayed peptides can be cleaved enzymatically or chemically to produce small-molecule-derivatized amino acids or peptide fragments. These amino acids or fragments are characterized by high-resolution mass spectrometry or NMR methods. Alternatively, if the compound is attached to the display peptide through a cleavable bond, the bond between the compound and the display peptide may be broken to generate compounds that are free of the amino acids from the display peptide. If desired, the structures of the isolated compounds can be compared to determine a consensus pattern for binding. This information can be used to further optimize the compounds in additional cycles of selection by generating libraries of cells and/or viruses displaying variants of the selected high affinity small molecules. Higher affinity molecules can be obtained by introducing mutations into the genes encoding the proteins responsible for the synthesis of the selected small molecules.

These methods may be generally applied to display small molecules produced by a variety of bacteria, yeast, and mammalian cells. In addition, novel compounds may be generated by mutating one or more enzymes or synthases in a particular biosynthetic pathway. For example, biotin analogs may be generated by mutating enzymes in the biotin biosynthetic pathway. Novel lipids may be produced by mutating fatty acid synthases or by mutating enzymes required for the myristilation, farnesylation, or geranylgeranylation of other proteins. Similarly, novel polyketides may be generated by mutating polyketide synthases.

Additional compounds of interest may be produced by expressing one or more heterologous proteins from a particular biosynthetic pathway in other organisms. For example, polyketide synthases from various bacteria, such as bacteria that naturally produce clinically relevant polyketide antibiotics, can be expressed in E. coli for the generation of hybrid polyketides with components produced by different heterologous polyketide synthases. DNA shuffling methods may also be used to combine nucleic acids encoding synthase domains from different bacteria and/or yeast to generate novel polypeptides, polyketides, and fatty acids.

A significant advantage of the present methods is the ability to culture the selected viruses or cells to generate an essentially unlimited supply of the selected compounds that bind the target molecule. For example, in one method of the invention, the selected viruses are used to infect additional cells, thereby generating additional viruses which display the desired small molecules. The viruses display novel small molecules on their surface and carry the genetic information necessary for the production of these small molecule in their genome. This allows one to screen very large numbers of viruses, each displaying a unique variant of a small molecule, while still being able to recover the genetic information that encodes the production of the selected small molecules. Viruses displaying a small molecule that interacts with a chosen target can be selectively captured by their affinity for that target (e.g., by biopanning). Subsequently, viruses can be amplified by cell infection to produce identical copies of the selected viruses. By repeating the process of selection and virus enrichment, with the latter obtained through infection of cells to produce identical copies of the selected viruses, small molecules with higher affinity for a target can be selected.

Alternatively, if a selected virus does not contain all of the nucleic acids responsible for the synthesis of the small molecule that it displays, the virus can be used to infect bacteria (e.g., a colony of identical bacteria) that contain the remaining or all of the nucleic acids required for the synthesis of the small molecule. This method allows the desired small molecule to be produced in the bacteria and then displayed on the surface of the viruses that are released from the infected bacteria.

In another possible method, cells are used to display variants of small molecules that have an affinity for a target. The cells display novel small molecules on their surface and carry within their genome (e.g., in a plasmid) the genetic information necessary for the production of these small molecule. This allows one to screen very large number of cells, each displaying a unique variant of a small molecule, while still being able to recover to the genetic information that encodes the production of the selected small molecules. Cells displaying a small molecule that interacts with a chosen target can be selectively captured by their affinity for that target (e.g., by biopanning) and then amplified by further growth. By repeating the process of selection and enrichment, with the latter obtained through growth of the selected cells, small molecules with higher affinity for a target can be selected.

Alternatively, one or more of the nucleic acids encoding enzymes involved in the synthesis of a selected compound may be isolated from a selected virus or cell (e.g., by polymerase chain reaction amplification) and transferred to another virus or cell, such as a commonly used laboratory strain, for the large-scale production of the selected compound. If desired, the isolation and transferring of the nucleic acids may be performed such that the nucleic acids are no longer operably linked to a nucleic acid encoding a surface protein. Thus, selected compounds may be expressed in soluble form and either secreted by the cells or isolated from cellular extracts.

Compounds generated using these methods may be used as therapeutic agents or may be used as lead compounds in the development of therapeutics for use in humans or animals of veterinary interest. For example, compounds that modulate the activity of an enzyme or the conductance of a transmembrane channel may be isolated and used as lead compounds. Additional rounds of selection may be used to optimize these compounds, resulting in compounds with increased affinity for the target molecule and decreased affinity for other molecules. The resulting therapeutic agents may be administered to subjects using standard methods. For example, the compounds may be administered with a pharmaceutically-acceptable diluent, carrier, or excipient, in unit dosage form. Methods well known in the art for making formulations are found in, for example, Remington: The Science and Practice of Pharmacy, (19th ed.) ed. A. R. Gennaro A R., 1995, Mack Publishing Company, Easton, Pa.

The following examples are provided to illustrate the invention. These examples may be readily adapted for the display of any compound of interest on the surface of bacteria, yeast, mammalian cells, or bacteriophage. They are not meant to limit the invention in any way.

### EXAMPLE 1

Display of Biotin and Biotin Analogs on Bacteriophage

Expression and Display of Biotinylated Protein Fusions

To generate a bacteriophage coat protein fusion that includes a display peptide to be modified by biotin (a 245 dalton molecule also known as vitamin H) or a biotin analog, a nucleic acid encoding an amino acid peptide that is recognized by the E. coli biotin ligase BirA is fused to the 5′ end of a nucleic acid encoding part or all of a pIII coat protein in a procedure analogous to that described by Fowlkes et al. (Biotechniques 3:422-428, 1992). Exemplary peptides that are biotinylated by BirA contain 23 amino acids (Schatz, Bio/Technology 11: 1138-1143, 1993) or 14 amino acids (Beckett et al., GLNDIFEAQKIEWH, SEQ ID NO.: 1; Protein Sci, 8:921-929, 1999). Other display peptides that may be used include all, or part of, a biotin carboxyl carrier protein such as a biotin carboxylase or decarboxylase. Examples of such display peptides include biotin carboxyl carrier protein (BCCP) from Pseudomonas aeruginosa (accession number AE004898), acetyl-CoA carboxylase, biotin carboxyl carrier protein from Vibrio cholerae (accession number AE004117), acetyl-CoA carboxylase (EC 6.4.1.2), biotin carboxyl carrier protein from Haemophilus influenzae (strain Rd KW20; accession number E64105), protein AccB from Pasteurella multocida (accession number AE006150), biotin carboxyl carrier protein from Synechococcus sp. (strain PCC 7942; accession number U59235), biotin carboxyl carrier protein from Aquifex aeolicus (accession numbers AE000736 and D70418), biotin carboxyl carrier protein from Anabaena sp. (accession number L14863), acetyl-CoA carboxylase subunit (biotin carboxyl carrier subunit) from Bacillus subtilis (accession number Z99116), biotin carboxyl carrier protein of acetyl-CoA carboxylase precursor from Arabidopsis thaliana (accession number AB005242), and putative acetyl-CoA carboxylase biotin carboxyl carrier protein from Neisseria meningitidis Z2491 (accession number AL162753).

To generate a coat protein fusion that contains the 23 amino acid display peptide, the nucleic acid 5′-C TCG AGA ATG GCT GGA GGC CTG AAC GAT ATT TTC GAA GCT CAG AAA ATC GAA TGG CAC GAG GAC ACT GGT GGC TCG TCTAGA-3′ (SEQ ID NO.: 2), which encodes the peptide MAGGLNDIFEAQKIEWHEDTGGS (SEQ ID NO.: 3) and contains XhoI and XbaI restriction enzyme cleavage sites at its 5′ and 3′ ends (underlined), is inserted between the XhoI and XbaI cloning sites in vector M655 which contains the tetracycline resistance gene derived from pBR322 (Fowlkes et al., supra). Alternatively, the display peptide may be expressed as a protein fusion containing all or part of the pVIII coat protein. Because a bacteriophage expresses a large number of pVIII proteins and the coat protein fusion makes only a relatively small portion of the bacteriophage coat, this fusion does not affect the bacteriophage assembly (Malik et al., Nucleic Acids Res. 25 (4):915-916, 1997).

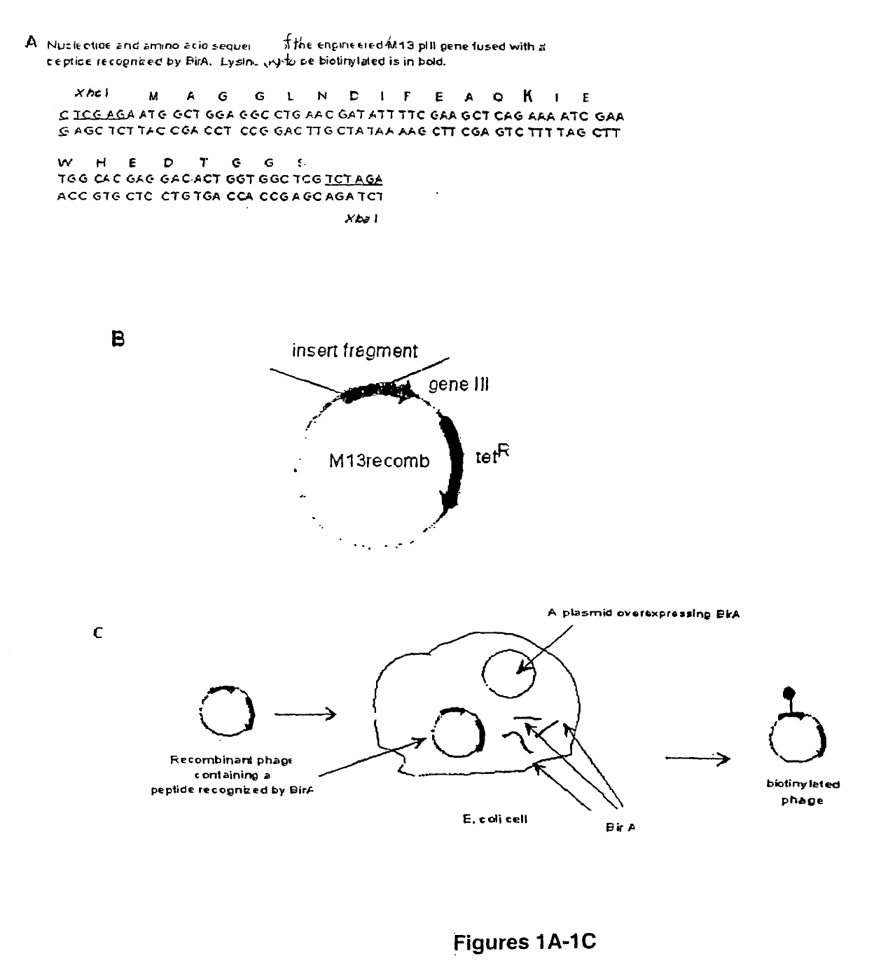

A nucleic acid encoding E. coli BirA, which covalently attaches biotin to the lysine residue in the display peptide described above (“K”), is obtained by polymerase chain reaction (PCR) amplification of chromosomal DNA of E. coli strain ATCC No. 11303 using Vent or Pfu DNA polymerases (Barker et al., J. Mol. Biol., 146:451-467, 1981; Howard et al., Gene 35:321-331, 1985). This BirA nucleic acid is placed under the regulation of a pTrc promoter in plasmid pTrcHis2 from Invitrogen (Tsao et al., Gene 169:59-64, 1996). Standard transformation techniques are used to insert the plasmid into an E. coli strain carrying an F′ episome (DH5α or TG1) that allows infection by a bacteriophage (see, for example, Ausubel et al., supra). The E. coli cells containing the plasmid are selected based on their resistance to ampicillin due to the ampicillin resistance gene in the plasmid. The selected E. coli are induced with IPTG to stimulate overexpression of BirA (FIG. 1). Cell transformation is followed by infection with a modified bacteriophage containing a 23 amino acid peptide sequence, fused to gene III, recognized by BirA protein to produce bacteriophage displaying biotin.

Expression of Biotinylated Protein Fusions Prior to Bacteriophage Assembly

The display peptide with the biotin modification may be co-expressed with BirA to ensure that the lysine residue in the display peptide is biotinylated Accordingly, a nucleic acid encoding the display peptide is fused to gene III in a vector which encodes a bacteriophage (e.g., pCANTAB5E from Amersham Pharmacia Biotech) that contains an amino acid substitution in gene II. Since the bacteriophage with this mutation requires a helper phage for bacteriophage assembly, this strategy allows as much time as needed for the biotinylation of the display peptide.

In particular, an E. coli strain is transformed with a plasmid that overexpresses BirA and a vector, which encodes a bacteriophage with a coat protein fusion. To ensure that the E. coli cells maintain both the vector encoding a bacteriophage and the BirA-expressing plasmid, these two constructs contain different antibiotic markers. Because the regulation of the coat protein fusion is under the control of the pLac promoter in the pCANTAB5E vector, BirA is preferably regulated under the control of a different promoter such as arabinoseBAD. The overexpression of BirA within the bacteria is induced with arabinose. Once the desired amount of display peptide has been biotinylated (e.g., after 30, 60, or 90 minutes), E. coli cells are infected with the helper phage M13K07 to produce bacteriophage displaying biotin.

An alternative procedure that can be used to maximize the amount of display peptide that is biotinylated by BirA involves using the same plasmid to co-express BirA and the coat protein fusion containing the display peptide. This procedure may be performed essentially as described previously using plasmid pDW363 (Tsao et al., supra). Briefly, overexpression of BirA and the coat protein fusion is induced by IPTG. After the coat protein fusion is biotinylated, E. coli cells are infected with bacteriophage to produce bacteriophage progeny that displays biotin on its surface. The bacteriophage used to infect the bacteria may be the bacteriophage described above which encodes a coat protein fusion or may be any other bacteriophage (e.g., encoding wild-type pIII). Even if the bacteriophage used to infect the bacteria encodes wild-type pIII protein instead of the coat protein fusion containing the display peptide, the large amount of overexpressed coat protein fusion that is encoded by the transformed plasmid effectively competes with the wild-type pIII coat protein encoded by the bacteriophage and is assembled into the bacteriophage progeny.

Synthesis and Display of Novel Biotin Analogs

To increase the amount and variety of biotin analogs synthesized by E. coli cells, one or more endogenous nucleic acids that encode proteins involved in the synthesis of biotin may be mutated to generate proteins with altered substrate specificity or catalytic efficiency (Example 8). Examples of enzymes involved in biotin synthesis that may be mutated include enzymes that are members of the following classes: 6.2.1.14, 2.3.1.47, 2.6.1.62, 6.3.3.3, and 2.8.1.6 (Marquet et al., Vitam. Horm. 61:51-101, 2001). A biotin ligase, such as BirA, may also be mutated to increase its ability to recognize the biotin analogs and use them to posttranslationally modify the display peptides. Alternatively, heterologous proteins from the biotin biosynthetic pathway of other organisms may be expressed in E. coli cells. The cells are then infected with a modified bacteriophage containing the coat protein fusion described above using standard procedures.

If this manipulation of the biotin biosynthetic pathway inhibits the growth of E. coli cells by decreasing the amount of naturally-occurring biotin, a duplicate copy of one or more enzymes required for the synthesis of biotin is introduced into E. coli, allowing E. coli to produce both naturally-occurring biotin and biotin analogs. Examples of enzymes involved in biotin synthesis that may be introduced into the E. coli include enzymes that are members of the following classes: 6.2.1.14, 2.3.1.47, 2.6.1.62, 6.3.3.3, and 2.8.1.6.

Detection, Selection, and Identification of Biotin Analogs that Bind a Target Molecule

The presence of biotin or biotin analogs on the surface of the bacteriophage may be detected based on the affinity of biotin for streptavidin. For example, streptavidin conjugated with an enzyme (e.g., alkaline phosphatase or horseradish peroxidase) is applied to a population of immobilized bacteriophage. The bacteriophage is washed to remove unbound or weakly bound streptavidin. Any streptavidin that remains bound to biotin or biotin analogs on the surface of the bacteriophage is detected based on the color or chemiluminescence produced by the reaction of the protein conjugated to streptavidin and a substrate.

Alternatively, bacteriophage expressing biotin or biotin analogs on their surface may be detected using streptavidin-coated magnetic beads and detected using an antibody against pVIII coat protein conjugated to alkaline phosphatase or horseradish peroxidase (Chaiet et al., Arch. Biochem. Biophys. 106:1-5, 1964; Bayer et al., Methods Enzymol. 184:49-51, 1990; Bayer et al., J. Chromatogr. 510:3-11, 1990; Brakel et al., Methods Enzymol. 184:437-442, 1990). Streptavidin mutants may be used to select biotin analogs with a desired binding affinity. The biotin and biotin analogs may be cleaved from the display peptide using standard methods and identified using standard mass spectrometry or NMR analysis.

### EXAMPLE 2

Display of Biotin on Bacteriophage

As discussed in Example 1, biotin was used as one example of organic molecules that can be displayed via a covalent attachment to a protein on the surface of a bacteriophage. The 322 amino acid BirA biotin ligase was used to immobilize biotin at a specific lysine residue on a display peptide expressed on the surface of bacteriophage (M13). In one exemplary approach, this was carried out as follows.

Experimental Details

To target the attachment of biotin onto an M13 coat protein, a protein fusion containing a M13 coat protein pIII and a 23-residue peptide that is biotinylated by BirA was generated. To ensure the availability of enough biotin and BirA, the cell media was supplemented with biotin, and BirA was overexpressed within the cells. TOP10F′ E. coli cells were selected as hosts for M13. These cells allowed both infection by M13 and regulation of an arabinose promoter to overexpress BirA.

Cloning of Peptide Recognized by BirA and Control Peptide not Recognized by BirA

To generate a bacteriophage coat protein fusion that includes a display peptide to be modified by biotin, a nucleic acid encoding a 33-residue peptide consisting of a 23-amino acid sequence recognized by BirA (MAGGLNDIFEAQKIEWHEDTGGS, Schatz P J, Bio/Technology 11: 1138-1143, 1993) followed by a hexahistidine tag (H)₆ (SEQ ID NO: 7) and a peptide recognized by the endoprotease Factor Xa (IEGR; SEQ ID NO: 8) was fused immediately after the signal peptidase cleavage site of geneIII of bacteriophage vector M13mp18 (New England Biolabs). An identical M13 vector, with the exception that the encoded lysine recognized by BirA was replaced by glycine, was used a control peptide. Each construct was obtained by ligating two fragments that were constructed as follows.

Fragment 1 was obtained by PCR amplification of M13mp18 using primers BspHI-FW (5′-GGT GCC TTC GTA GTG GCA TTA CGT ATT TTA CCC-3′, SEQ ID NO: 9) and Biopep-1-OUTSIDE-BK (5′-TTC GAA AAT ATC GTT CAG GCC TCC AGC CAT GGA GTG AGA ATA GAA AGG AAC AAC TAA AGG AAT TGC GAA TAA-3′, SEQ ID NO: 10). The resulting fragment 1 was purified and further amplified using primers BspHI-FW (5′-GGT GCC TTC GTA GTG GCA TTA CGT ATT TTA CCC-3′, SEQ ID NO: 11) and Biopep-1-INSIDE-BK (5′-Phos-GTG CCA TTC GAT TTT CTG AGC TTC GAA AAT ATC GTT CAG GCC TCC AGC CAT-3′, SEQ ID NO: 12). This fragment was named Fragment-1-FINAL. Fragment 2 was obtained by PCR amplification of M13mp18 using primers AlwNI-BK (5′-AAG CCA GAA TGG AAA GCG CAG TCT CTG AAT TTA C-3′, SEQ ID NO: 13) and Biopep-1-OUTSIDE-FW (5′-CAC CAT CAC ATC GAG GGA AGG GCT GAA ACT GTT GAA AGT TGT TTA GCA AA CCC CA-3′, SEQ ID NO: 14). The resulting fragment 2 was purified and further amplified using primers AlwNI-BK (5′-AAG CCA GAA TGG AAA GCG CAG TCT CTG AAT TTA C-3′, SEQ ID NO: 15) and Biopep-1-INSIDE-FW (5′-Phos-GAG GAC ACT GGT GGC TCG CAT CAT CAT CAC CAT CAC ATC GAG GGA AGG GCT-3′, SEQ ID NO: 16). This fragment was named Fragment-2-FINAL. Fragment-1-FINAL and Fragment-2-FINAL were ligated, and a fragment of the desired size was isolated. This isolated fragment was digested with BspHI and AlwNI and ligated between the same sites of M13mp18. The control peptide was produced in an identical manner with the exception that primer Biopep-1-INSIDE-BK was replaced with Biopep-1-INSIDE-BK-CNTRL in all of the steps mentioned above. These constructs were verified by DNA sequencing.

Cloning of BirA

A nucleic acid encoding E. coli BirA was obtained by polymerase chain reaction (PCR) amplification of chromosomal DNA of E. coli strain ATCC No. 11303 using Pfu DNA polymerase from Stratagene (Barker et al., J. Mol. Biol., 146:451-467, 1981; Howard et al., Gene 35:321-331, 1985). This BirA nucleic acid was placed under the regulation of a pBAD promoter. The gene was inserted between the XhoI and HindIII sites of the plasmid pBADHISA (Invitrogen). The resulting vector was named pBAD-BirA‘2’. The primers used in the vector construction were BirA-FW (5′-TAT AGA TAC CCA TGG GTA TGA AGG ATA ACA CCG TGC CAC TG-3′, SEQ ID NO: 17 containing a XhoI cleavage site) and BirA-BK (5′-ATC ATC ACG AAG CTT TTA TTT TTC TGC ACT ACG CAG GGA TAT-3′, SEQ ID NO: 18 containing a HindIII cleavage site).

Host Cells

The host strain TOP10F′ (Invitrogen) was grown in 2× TY and 15 ug/ml of tetracycline. This strain was transformed with pBAD-BirA‘2’ and selected in 15 ug/ml of tetracycline and 100 ug/ml of ampicillin at 37° C. The BirA gene was always induced using a stock of 20% arabinose to give a final concentration of 0.2%.

Cell Growth and Bacteriophage Infection

To prepare TOP10F′pBADBirA‘2’ cells for phage infection, cells were grown overnight (˜16 hours) in the presence of biotin at a concentration of 100 μg /ml plus 15 μg/ml of tetracycline and 100 μg/ml of ampicillin in ˜10 ml 2× TY at 37° C. Then, 0.25 ml of cells grown overnight were added to 10 ml of fresh 2× TY, 100 μg/ml biotin, 15 μg/ml of tetracycline, and 100 μg/ml of ampicillin and grown until the optical density at 600 nm reached 0.5. Cells were then induced with arabinose at a final concentration of 0.2% arabinose and grown for 30 minutes. Next, 0.25 ml of induced cells plus ˜10⁵ bacteriophage particles encoding the peptide recognized by BirA (“bacteriophage K”) or not (“bacteriophage G”) were added separately to 10 ml of 2× TY containing 0.2% arabinose, 15 μg/ml of tetracycline, 100 μg/ml of ampicillin, and 100 mg/ml biotin. These cultures were grown for 6 hours, and then supernatants containing the bacteriophages were collected.

Bacteriophage Titers and Infection

The titers of bacteriophage K and bacteriophage G were determined to be 2-5×10¹⁰ pfu/ml using the host strain TG1 that contains an F′ epitope that allows bacteriophage infection. To be able to compare the results obtained from binding experiments using bacteriophage K and bacteriophage G, these bacteriophage were treated with the endoprotease Factor Xa prior to infection. This enzyme recognizes the sequence IEGR (SEQ ID NO: 8) and cleaves after the “R,” thereby removing the insert added after the signal peptidase site of pIII, making bacteriophage K and bacteriophage G identical. This strategy was used only to count the number of bacteriophage K and bacteriophage G particles. The treatment of bacteriophage K and bacteriophage G with Factor Xa prior to infection was observed to increase infection. This result is summarized in the experiment below.

A 50 μl aliquot of phage from a 1/10⁶ dilution of a stock of bacteriophage K or bacteriophage G was incubated separately in 100 mM NaCl, 2 mM CaCl₂, and 10 mM Tris-HCl pH 8.0, and used to infect TG1 cells. Samples that were incubated with Factor Xa included 2 μg of Factor Xa in a total volume of 52 μl.

K=Phage displaying peptide recognized by BirA

G=Phage displaying peptide not recognized by BirA

K without Factor Xa=187 bacteriophage

K with Factor Xa=477 bacteriophage

G without Factor Xa=23 bacteriophage

G with Factor Xa=239 bacteriophage

Based on these data, Factor Xa was shown to have a favorable effect on infection, especially for bacteriophage G. Since the effect occurred for both bacteriophage G and K, the effect was likely caused by the interaction of Factor Xa with bacteriophage proteins rather an effect of Factor Xa on the bacterial host cells. Since K and G are identical with the exception of the amino substitution K→G in the fusion protein, the removal of the peptide inserted after the peptidase cleavage signal of pIII clearly improved infection.

Biotin Display

For the display of biotin on the surface of bateriophage, host cells were grown as described above. Six hours after induction, cultures were centrifuged at about 7000×g, and the supernatant was saved. Then, 20 μl of a 10⁴ dilution of bacteriophage K or G in 10 mM Tris-HCl pH8.0 were incubated separately with 5 μl (˜15 pmoles) of streptavidin-coated beads (Dynal) and 175 μl of 10 mM Tris-HCl pH 8.0 with shaking at 1,400 rpm for 30 minutes. Next, 200 μl of 10 mM Tris-HCl pH 8.0 and 0.1% Nonident P-40 was added, and the beads were collected. The beads were washed three times with 500 μl of 10 mM Tris-HCl pH 8.0 and 0.1% Nonident P-40. The beads were then suspended in 200 μl 10 mM Tris-HCl pH 8.0, 0.1% Nonident P-40, and 2 mM CaCl2, and then 4 μl (4 μg) of Factor Xa was added. Factor Xa was used to cleave the coat protein fusion to separate the bacteriophage bound to the streptavidin-coated beads from the beads, so that the number of bacteriophage that had displayed biotin and bound the strepavidin-coated beads could be determined. The reaction was incubated overnight (˜16 hours), and then the eluted bacteriophage were plated using TG1 cells from an overnight culture. The number of phage that were able to infect TG1 cells after binding to streptavidin-coated beads are listed below in Table I; these numbers represent the number of bacteriophage that displayed a sufficient amount of biotin for the bacteriophage to bind streptavidin-coated beads.

TABLE I
Percentage of Bacteriophage Bound to Streptavidin-coated Beads

| | # of phage | | |
|---|---|---|---|
| | used to bind to | # of bound phage | |
| | stv-coated beads | able to bind TG1 cells | % bound |
| K | 96,500 | 1075 | 1.113 |
| G | 97,500 | 13 | 0.013 |

These results indicated that bacteriophage K is clearly preferably immobilized by binding to streptavidin-coated beads compared to the much lower extent of immobilization of bacteriophage G to the beads. These results suggest that approximately 1% of bacteriophage K displays biotin.

Effect of the Exogenous Addition of Biotin on Biotinylation

To measure the effect of exogenously added biotin on biotinylation, the extent of biotinylation in the experiment above was measured by growing cells with and without biotin. For comparison, the results with and without biotin for bacteriophage K are shown.

TABLE II
Percentage of Bacteriophage Incubated with or without Biotin
that were able to Bind Stv-Coated Beads

| | | # of bound phage | % bound |
|---|---|---|---|
| | | used to bind to | able to bind |
| | # of phage | stv-coated beads | TG1 cells |
| K with biotin | 96,500 | 1075 | 1.113 |
| K without biotin | 109,000 | 850 | 0.780 |

These results show that the addition of exogenous biotin to the culture medium increased the amount of biotinylated bacteriophage K by approximately 43%. Most likely, this exogenous biotin diffuses into the host cells and biotinylates the display peptide in vivo, resulting in an increased percentage of biotinylated display peptide that is incorporated into the coat of the bacteriophage.

Purification of Bacteriophage K and G using Stv-38

As discussed above, elution of biotinylated bacteriophage K from streptavidin-coated beads can be performed by cleaving the coat protein fusion with Factor Xa to separate the biotinylated display peptide from the rest of the bacteriophage particle. An alternative procedure that utilizes the modified streptavidin Stv-38, which has a ˜10⁸ M⁻¹ affinity for biotin, was also developed. To reduce the amount of biotin that is present in the supernatant that contains bacteriophage K and G, 1 ml of bacteriophage K and G was precipitated with 200 μl of 20% PEG 8000 in 2.5 M NaCl. The bacteriophage were resuspended in 1 ml of 20 mM Tris pH 7.4 and 150 mM NaCl. Approximately, 10 μg of Stv-38 in 200 μl was added to microtiter wells and incubated for 48 hours. Then, plates were washed four times with 20 mM Tris pH 7.5 and 150 mM NaCl and then blocked with 3% BSA (low fatty acid content 0.002%) in 20 mM Tris pH 7.5 and 150 mM NaCl for three hours. Then, plates were washed four times with 20 mM Tris pH 7.5, 150 mM NaCl and 0.1% Nonident P-40. A 20 μl aliquot of a 1/100 or 1/1000 dilution of K or G was added to each well in a total reaction volume of 200 μl in which 180 μl were 20 mM Tris pH 7.5, 150 mM NaCl, and 0.1% Nonident P40. The reactions were incubated for one hour, and then unbound bacteriophage were removed by washing the microtiter wells four times with 20 mM Tris pH 7.5, 150 mM NaCl, and 0.1% Nonident P40. Bound bacteriophage were eluted by incubation in 20 mM Tris pH 7.5, 150 mM NaCl, 0.1% Nonident P40, 3 mM biotin, and 2 mM CaCl₂ for one hour in 250 μl. Eluted bacteriophage were incubated for 17 hours with 1.5 μl of Factor Xa. Then, different amounts of phage were mixed with TG1 cells and plated. These results are shown in Table III.

TABLE III
Percentage of Bacteriophage that Displayed a Sufficient
Amount of Biotin to Bind Stv-38

| | Input | Bound | Ratio (%) |
|---|---|---|---|
| K (1/100 dilution) | 250,000 | 2130 | 0.852 |
| K (1/1000 dilution) | 25,000 | 126 | 0.504 |
| G (1/100 dilution) | 1,590,500 | 27 | 0.002 |
| G (1/1000 dilution) | 159,500 | 7 | 0.004 |

These results show that immobilized Stv-38 can be successfully used for purification of bacteriophage displaying biotin on their surface.

Summary

These above results demonstrated that a peptide, termed “K,” can be displayed on the surface of M13 and modified with the small molecule biotin. As a control, another peptide, termed “G,” which is identical to peptide “K” with the exception that the lysine which is biotinylated is replaced with glycine, was also displayed on the surface of M13. This control peptide “G” was not biotinylated. In contrast, approximately 1% of the “K” peptides were biotinylated.

In certain cases, this percentage may be relatively low due to the fast turnover of M13, which is approximately 5 minutes. If desired, this turnover can be slowed down by using a phagemid that requires a helper phage for the production of a mature M13 bacteriophage. This phagemid also contains a fusion between pIII and the peptide recognized by BirA. Using this construct, it is possible to incubate the pIII-peptide recognized by BirA as long as necessary to achieve full biotinylation. Once achieved, a helper phage is added to begin the assembly and release of M13, fully biotinylated, from the cells. One example of such a vector useful for this purpose is pCANTAB5 E (RPAS Expression module, Amersham). If this vector is utilized, it is necessary to change the antibiotic resistance of the vector containing the BirA gene. In particular examples, chloramphenicol resistance or tetracycline resistance genes may be used in place of the ampicillin resistance gene present in pBADBirA‘2’.

Additionally, the above results demonstrate the ability to capture biotinylated peptides attached to M13 with natural streptavidin-coated beads. Since the binding of biotin to natural streptavidin is essentially irreversible, bound M13 was recovered using Factor Xa. This enzyme cleaves after the peptide sequence IEGR (SEQ ID NO: X) that is present at the C-terminus of peptides “K” and “G,” and thus separates the biotinylated display peptide bound to the Streptavidin-coated beads from the rest of the bacteriophage particle allowing the bacteriophage to be purified from the beads. In the absence of Factor Xa, M13 was still able to replicate. As an alternative to using Factor Xa, a variant of natural streptavidin (Stv-38) was used to bind and release biotinylated bacteriophage under mild conditions. The results described herein also demonstrate that the ability of M13 to infect TG1 cells is increased if the amino end of pIII containing peptides “K” or “G” is removed using Factor Xa.

Similar methods can be used to display other molecules on the surface of bacteriophage.

### EXAMPLE 3

Display of Biotin on a Bacillus subtilis Spore

Biotin was also displayed on the surface of a Bacillus subtilis spore. To target the attachment of biotin to a Bacillus spore protein, a protein fusion between CotB, a spore outer coat protein, and a display peptide that is biotinylated by BirA was produced. The nucleic acid fusion between the CotB gene and the DNA encoding the display peptide recognized by BirA was placed under the regulation of the CotB promoter. This nucleic acid fusion was cloned into a vector that recombines in a double-crossover event at the AmyE locus of the Bacillus chromosome. To ensure the availability of the E.coli BirA gene product, the BirA gene was expressed under the regulation of the hybrid IPTG-inducible spac promoter. This construct was also inserted via a double crossover recombination event at the LacA locus of the same Bacillus chromosome. Therefore, two chromosomal insertions were produced using these constructs. The recombination events at the AmyE and LacA loci conferred resistance to chloramphenicol and erythromycin, respectively.

Cloning of BirA

Plasmid pA-spac was used to express E.coli BirA. Since this plasmid lacks a ribosomal binding site (RBS) for the production of BirA, the RBS described by Yansura and Henner (Proc Natl Acad Sci U S A 81(2):439-443, 1984) was added to this vector. This construct was produced by amplifying two fragments by PCR using pA-spac as a template. The first fragment was amplified using the primers Frag1-BK (5′-ATC ATA CAT GAA TTC TAG ATA CAC CTC CTT AAG CTT AAT T-3′, SEQ ID NO: 19) and Frag1-FW (5′-TTT ATG CAG CAA TGG CAA GAA CGT CC-3′, SEQ ID NO: 20). The second fragment was amplified using the primers Frag2-FW (5′-TAT CTA GAA TTC ATG ATC TAG AGT CGA CCT GCA GGC ATG C-3′, SEQ ID NO: 21) and Frag2-BK (5′-AAC CCT GAT AAA TGC TTC AAT AAT ATT GAA AAA GGA AGA-3′, SEQ ID NO: 22). Fragments 1 and 2 were digested with the restriction enzyme EcoRI and subsequently ligated using T4 DNA ligase. This fragment and pA-spac were subsequently digested using the restriction enzyme SacI. The larger fragment of pA-spac was recovered and ligated to the fragment resulting from the ligation of fragments 1 and 2. Clones with the desired orientation were selected using the restriction enzymes PacI and EcoRI. The resulting vector was named pA-spac-RBS. The gene encoding BirA was amplified from E.coli using the primers BirA-EcoRI-FW (5′-TAT CTA GAA TTC ATG AAG GAT AAC ACC GTG CCA CTG AAA T-3′, SEQ ID NO: 23) and BirA-SphI-BK-MOD (5′-AGT TTG AAG CAT GCT TAT TTT TCT GCA CTA CGC AGG GAT A-3′, SEQ ID NO: 24). The amplified fragment was cloned between the EcoRI and SphI sites of pA-spac-RBS. The resulting vector was labeled “pA-spac-RBS-BirA.” All PCR reactions were carried out using Pfu polymerase from Stratagene, and all restrictions enzymes and T4 DNA ligase were from New England Biolabs.

CotB Fusions

Three constructs that contain a fusion between the CotB gene of B. subtilis and a nucleic acid encoding a peptide that contains a 23-amino acid sequence recognized by BirA (MAGGLNDIFEAQKIEWHEDTGGS, SEQ ID NO: 25, Schatz, Bio/Technology 11:1138-1143, 1993) were designed. Four residues that are recognized by the endoprotease Factor Xa (IEGR SEQ ID NO: 8) were added at the amino end of the encoded protein fusion. Also added at the C-terminus of this sequence were five residues that are recognized by the endoprotease Enterokinase, light chain (DDDDK SEQ ID NO: 26). This peptide is denoted “peptide-K.” As a control, a similar peptide with a glycine in place of the lysine biotinylated by BirA was designed. This peptide is denoted “peptide-G.”

Six fusions were produced, three involving peptide-K and three involving peptide-G. For a set of fusions (Fusion 1), peptide-K and peptide-G were added to the C-terminus of CotB following residue 275 of Cot B. For another set of fusions (Fusion 3), the last 41 amino acids of CotB were also added to the C-terminus of Fusion 1. In the last set of fusions (Fusion 2), peptide-K and peptide-G were fused to the first 275 amino acids of CotB. For Fusion 1, a fragment of CotB DNA was PCR-amplified from B. subtilis chromosome using primers B1-SphI-FW (5′-ATC GAC ATG CAT GCA CGG ATT AGG CCG TTT GTC C-3′, SEQ ID NO: 27) and B3-BglII-BK (5′-TAG TAG AAA GAT CTG GAT GAT TGA TCA TCT GAA GAT TTT AGT GA-3′, SEQ ID NO: 28). Similarly, a template for producing peptide-K was obtained by annealing primers PEP-FW (5′-ATC CTA ATC TCG AGA ATG GCT GGA GGC CTG AAC GAT ATT TTC GAA GCT CAG AAA ATC GAA TGG CAC GAG GAC ACT GGT-3′, SEQ ID NO: 29) and PEP-BK (5′-ATA CTA ATC ACC GGT GCG ACC CTC GAT GTG ATG GTG ATG ATG ATG CGA GCC ACC AGT GTC CTC GTG CCA TTC GAT-3′, SEQ ID NO: 30) and extending in the presence of Pfu polymerase for one cycle. The resulting product was denoted “template-K.” The template for producing peptide-G was obtained by annealing primers PEP-FW-CTRL (5′-ATC CTA ATC TCG AGA ATG GCT GGA GGC CTG AAC GAT ATT TTC GAA GCT CAG GGT ATC GAA TGG CAC GAG GAC ACT GGT-3′, SEQ ID NO: 31) and PEP-BK (5′-ATA CTA ATC ACC GGT GCG ACC CTC GAT GTG ATG GTG ATG ATG ATG CGA GCC ACC AGT GTC CTC GTG CCA TTC GAT-3′, SEQ ID NO: 32) and extending in the presence of Pfu polymerase for one cycle. The resulting product was denoted “template-G.” DNA encoding peptide-K and peptide-G was obtained using template-K and template-G, respectively, by PCR using primers Bio-BglII-FW (5′-TAG TAG AAA GAT CTA TCG AGG GAA GGA TGG CTG GAG GCC TGA ACG ATA TTT TCG AAG CTC AG-3′, SEQ ID NO: 33) and Bio-SalI-BK (5′-ATA GTA GCG TCG ACT TAT TTA TCA TCA TCA TCC GAG CCA CCA GTG TCC TCG TGC CAT TCG AT-3′, SEQ ID NO: 34). DNA encoding peptide-K, peptide-G, and the amplified CotB fragment were digested with the restriction enzyme BglII. The purified CotB fragment was subsequently ligated with the DNA encoding peptide-K or peptide-G, separately. These ligations produced two fragments named “Fusion1K,” and “Fusion1G.” Fusion1K and fusion1G were cloned separately between the SalI and SphI restriction sites of vector pDG364. The resulting vectors were named pDG364-fusion1K and pDG364-fusion1G, respectively.

For Fusion 2, the construction of another peptide-K/peptide-G—CotB fusion was achieved in three steps. First, primers B1-SphI-FW (5′-ATC GAC ATG CAT GCA CGG ATT AGG CCG TTT GTC C-3′, SEQ ID NO: 35) and B6-BglII-BK (5′-TAG TAG AAA GAT CTC ATT CAA ATT CCT CCT AGT CAC TTA TAC ATA-3′, SEQ ID NO: 36) were utilized to amplify CotB DNA by PCR amplification of a region of the B. subtilis chromosome. This fragment is denoted fragment “1.” Peptide-K and peptide-G were PCR-amplified from template-K and template-G, respectively, using the primers Bio-BglII-FW-Fusion2 (5′-TAG TAG AAA GAT CTA TGA TCG AGG GAA GGA TGG CTG GAG G-3′, SEQ ID NO: 37) and Bio-XhoI-BK (5′-ATA GTA GCC TCG AGT TTA TCA TCA TCA TCC GAG CCA CCA GTG T-3′, SEQ ID NO: 38). These amplifications produced fragments named “2K”, and “2G.” Lastly, primers B7-XhoI-FW-MOD (5′-AGT AGT AAC TCG AGA TGA GCA AGA GGA GAA TGA AAT ATC A-3′, SEQ ID NO: 39) and B3-SalI-BK (5′-TAG TAG AAG TCG ACT TAG GAT GAT TGA TCA TCT GAA GAT TTT AGT GA-3′, SEQ ID NO: 40) were used to amplify a third fragment named “3.” Fragments 1, 2K, and 2G were digested with BglII, and then fragment 1 was ligated separately with fragments 2K and 2G to produce 12K and 12G, respectively. Subsequently, fragments 12K, 12G, and 3 were digested with XhoI. Then, fragment 3 was ligated with 12K and 12G separately to produce fragments named “Fusion2K” and “Fusion2G.” Fusion2K and fusion2G were cloned separately between the SalI and SphI restriction sites of vector pDG364. The resulting vectors were named pDG364-fusion2K and pDG364-fusion2G, respectively.

To generate Fusion 3, fusion1K and fusion1G were used as a template with primers B1-SphI-FW (5′-ATC GAC ATG CAT GCA CGG ATT AGG CCG TTT GTC C-3′, SEQ ID NO: 41) and Bio-XhoI-BK (5′-ATA GTA GCC TCG AGT TTA TCA TCA TCA TCC GAG CCA CCA GTG T-3′, SEQ ID NO: 42). This amplification produced two fragments denoted “3K” and “3G.” Then, primers B5-XhoI-Fw (5′-AGT TGA AAC TCG AGG ATT ATC AAT CAT CAA GAT CAC CAG GC-3′, SEQ ID NO: 43) and B4-SalI-BK (5′-AGT TGA AAG TCG ACT TAA AAT TTA CGT TTC CAG TGA TAG TCT ATC GT-3′, SEQ ID NO: 44) were used to obtain the last 41 residues of CotB from chromosomal B. subtilis DNA by PCR amplification. This fragment was named “41.” Subsequently, fragments 3K, 3G, and 41 were digested with XhoI. Then, fragment 41 was ligated separately with 3K or 3G to produce fragments named “Fusion3K” and “Fusion3G.” Fusion3K and fusion3G were cloned separately between the SalI and SphI restriction sites of vector pDG364. The resulting vectors were named pDG364-fusion3K and pDG364-fusion3G, respectively.

Chromosomal Insertions

The B. subtilis host strain PY79 was used for display of biotin. Competent cells were obtained using the “Groningen method” (Method 3.2 in “Molecular Biological Methods for Bacillus” Edited by Harwood and Cutting, Wiley-Interscience 1990). Vector pA-spac-RBS-BirA was linearized using NgoM IV and inserted via a double crossover recombination event in the Bacillus chromosome. Transformants were selected on erythromycin plates. Colonies that grew on erythromycin plates were screened using the primers ON4-complementMOD (5′-GTG GCA CAT TTC AAA CGA ATA CG-3′, SEQ ID NO: 45) and ON5-complement (5′-GCT CAA CTC CAA ATA TAG CTT GAA-3′, SEQ ID NO: 46). A positive clone was selected and named “PY79-BirA.” This strain was subsequently used to make competent cells. The vectors pDG364-fusion1K, pDG364-fusion1G, pDG364-fusion2K, pDG364-fusion2G, pDG364-fusion3K, and pDG364-fusion3G were linearized with PstI. The large fragment resulting from the digestion with PstI was used in the transformations. Positive clones were labeled as PY79-BirA-1K, PY79-BirA-1G, PY79-BirA-2K, PY79-BirA-2G, PY79-BirA-3K, and PY79-BirA-3G, respectively. Transformants were selected on chloramphenicol plates. Colonies that grew on chloramphenicol plates were screened using the primers AmyS (5′-CCA ATG AGG TTA AGA GTA TTC C-3′, SEQ ID NO: 47) and AmyA (5′-CGA GAA GCT ATC ACC GCC CAG C-3′, SEQ ID NO: 48). All constructs were verified by DNA sequencing.

Spore Formation-Biotinylation in vivo

Spores were obtained using “the nutrient exhaustion method” (Method 9.1 in “Molecular Biological Methods for Bacillus,” Edited by Harwood and Cutting, Wiley-Interscience, 1990). This method was carried out as previously described; however, solutions were supplemented with 1 mM IPTG and 100 μM of biotin during the solid and liquid stages of spore formation. Spores were collected 24 hours following T₀ (the start of sporulation) and purified by lysozyme treatment and by salt and detergent washes as described on procedure 9.8.2 on pages 415-416 in “Molecular Biological Methods for Bacillus” (supra).

To test if spores derived from PY79-BirA-1K, PY79-BirA-1G, PY79-BirA-2K, PY79-BirA-2G, PY79-BirA-3K, and PY79-BirA-3G were able to display biotin, the spores were incubated with approximately 5 μl of streptavidin-coated magnetic beads (Dynal) for one hour. Spores were washed four times in 10 min intervals with 500 μl of 150 mM NaCl, 20 mM Tris-HCl pH 7.4, and 0.1% of Nonident-P-40. Finally, beads were resuspended in 1 ml of 150 mM NaCl, 20 mM Tris-HCl pH 7.4, and 0.1% Nonident P-40, and aliquots (10 μl, 100 μl, and 800 μl) were mixed with LB top agar and plated on pre-warmed LB plates. It was observed that the number of Bacillus colones that grew on the plate that had 10 μl and 100 μl were proportional to each other; however, a strong inhibition for Bacillus growth on the plate that had the largest aliquot was always seen. This happened wth all six constructs tested. The results (from the 100 μl aliquot) are shown in Table IV below.

TABLE IV
Ratio of Spores Bound to Streptavidin-coated Beads

| | Input (#) | Out (#) | Ratio (%) |
|---|---|---|---|
| PY79-BirA-K1 | 1,075,900 | 300 | 0.0279 |
| PY79-BirA-G1 | 843,900 | 100 | 0.0118 |
| PY79-BirA-K2 | 10,000 | 920 | 9.2 |
| PY79-BirA-G2 | 10,000 | 0 | 0 |
| PY79-BirA-K3 | 919,300 | 260 | 0.0283 |
| PY79-BirA-G3 | 814,900 | 240 | 0.0049 |

In this table, “#” refers to the total number of spores measured as an input and bound spores to streptavidin magnetic beads. These results indicate that spores derived from PY79-BirA-K2, which has the peptide-K located at the amino end of CotB can be biotinylated more efficiently than the other two constructs in which peptide K is located near the C-terminus of CotB.

Biotinylation in vitro

To determine whether biotinylation of PY79-BirA-K1 and PY79-BirA-K3 can be enhanced in vitro, spores expressing the display peptide were incubated in a medium containing biotin and BirA to measure the extent of biotinylation of the display peptide in vitro. To produce BirA for this assay, expression from the BirA expression vector TOP10F′pBADBirA‘2’ was induced in cells. Four hours after the induction of the arabinose promoter, cells were resuspended in 1/10 the culture volume and incubated in 50 mM Tris-HCl, pH 8.0 with 50 μg/ml of lysozyme for one hour to release BirA into the culture medium. Then, aliquot of spores (10⁵-10⁶) were incubated with 20 μl of BirA, 10 mM MgCl₂, 50 mM KCl, 20 μM biotin, 3 mM ATP, 0.1% BSA, and 0.1 mM DTT as described by Polyak et al. (J Biol Chem. 276:3037-45, 2001). The results of this assay are shown in Table V below.

TABLE V
Ratio of Spores Bound to Streptavidin-Coated Beads

| | Input | Out | Ratio (%) |
|---|---|---|---|
| PY79-BirA-K1 | 919,600 | 30,000 | 3.262 |
| PY79-BirA-G1 | 417,600 | 1250 | 0.299 |
| PY79-BirA-K3 | 176,900 | 125 | 0.071 |
| PY79-BirA-G3 | 234,900 | 250 | 0.106 |

These results indicate that for some display peptides, biotinylation can be enhanced, if desired, by incubating the bacteria expressing the display peptides in a medium containing biotin and BirA to allow in vitro biotinyation.

Summary

Several fusion proteins containing a display peptide with a BirA recognition sequence were biotinylated in vivo and expressed on the surface of B. subtilis spores. Similar methods can be used to display other molecules on the surface of bacteria, such as B. subtilis spores.

### EXAMPLE 4

Display of Fatty Acids on Bacteriophage

For the display of fatty acids on bacteriophage, a small acidic protein responsible for acyl group activation in fatty acid biosynthesis, called acyl carrier protein (ACP), is expressed on the surface of a bacteriophage as part of a coat protein fusion. ACP undergoes a posttranslational modification in which the 4′-phosphopantetheine group from CoA is transferred by holo-ACP-synthetase to a specific serine of apo-ACP. This 4′-phosphopantetheine modification contains a free sulfhydryl group that binds fatty acids via a thioester linkage.

The fatty acids are produced by E. coli using the endogenous fatty acid pathway. Only the fatty acids that are synthesized on ACP contained in the coat protein fusion are transported to the bacteriophage coat protein. In contrast, fatty acids that are synthesized on any of the approximately 60,000 copies of endogenous E. Coli ACP remain inside E. coli and are not incorporated into the bacteriophage coat protein because endogenous ACP molecules are not part of the coat protein fusions. Because only ˜100-200 bacteriophage infect each cell and each bacteriophage contains only ˜5 copies of the ACP-coat protein fusion, only approximately 500-1000 fatty acids molecules per cell modify coat protein fusions instead of endogenous ACP molecules. Thus, the incorporation of fatty acids into the bacteriophage coat protein is expected to have minimal, if any, adverse effect on the cell cycle of E. coli.

Expression and Display of Fatty Acids

For the generation of a nucleic acid encoding an ACP-coat protein fusion, a nucleic acid encoding ACP is PCR amplified from E. coli genomic DNA, yeast genomic DNA, plant genomic DNA, or any other appropriate source (Rawlings et al., J. Biol. Chem. 267:5751-5754, 1992). This nucleic acid encoding ACP is fused to the bacteriophage gene III as described previously (Fowlkes et al., supra). If necessary, a linker encoding a recognition sequence for Factor Xa (Ile-Glu-Gly-Arg, Ile-Asp-Gly-Arg, or Ala-Glu-Gly-Arg; SEQ ID NOS: 4-6, respectively) can be inserted between the ACP nucleic acid and gene III (Nagai et al., Nature 309:810-812, 1984). This linker allows the cleavage of the ACP-fatty acid complex from the bacteriophage coat protein.

An E. coli strain is infected with a bacteriophage that encodes an ACP-coat protein fusion, in which ACP is an endogenous or an heterologous protein (e.g., E. coli ACP or a heterologous ACP such as spinach ACP). E. coli cells are grown in the presence of antibiotics to select those retaining the vector encoding a bacteriophage. Pantothenate (e.g., 1-100 mM) is added to the media to minimize the release of the 4′-phosphopantetheine cofactor attached to the ACP-coat protein fusion and thereby increase the amount of phosphopantetheinylated protein fusion that may be modified with a fatty acid (Keating et al., J. Biol. Chem. 270:22229-22235, 1995). In particular, panthothenate inhibits the enzyme ACP phosphodiesterase which would otherwise hydrolyze the 4′-phosphopantetheine cofactor from ACP. To minimize the release of the fatty acids attached to the ACP-coat protein fusion, the antiproliferative agent didemnin B (e.g., 1-100 mM) is also added to the media to uncompetitively inhibit palmitoyl protein thioesterase (Meng et al., Biochemistry 37:10488-10492, 1998).

To further increase the amount of ACP-coat protein fusion that is modified by the addition of 4′-phosphopantetheine, a gene encoding an ACP-synthase, such as E. coli ACP-synthase (dpj) (Lambalot et al., J. Biol. Chem. 270:24658-24661, 1995) may be optionally obtained and overproduced in E. coli, as described by Lambalot and Walsh (Lambalot et al., supra). If a heterologous ACP (e.g., spinach ACP) is used as part of the ACP-coat protein fusion, it may be phosphopantheinylated by endogenous E. coli ACP-synthase that is or is not overexpressed. Alternatively, a heterologous ACP-synthase (e.g., Brassica napus ACP-synthase) may be expressed in the bacteria to increase the amount of ACP-coat protein fusion that is phosphopantheinylated (Guerra et al., J. Biol. Chem. 263:4386-4391, 1988). The use of an ACP-coat protein fusion containing a heterologous ACP may be preferable to the use of an ACP-coat protein fusion containing an E. coli ACP if the E. coli ACP-coat protein fusion is found to inhibit cell growth.

Expression of Modified Protein Fusion Prior to Bacteriophage Assembly

An alternative method to increase the amount of ACP-coat protein fusion that is modified with a fatty acid involves the use of a vector that encodes a bacteriophage that requires a helper phage for bacteriophage assembly. This approach ensures sufficient modification of the ACP with fatty acids in the protein fusion prior to bacteriophage assembly. By expressing the ACP-coat protein fusion in bacteria in the absence of helper phage, the ACP-coat protein fusion is produced in an amount sufficient to compete, as an immobilization support in fatty acid synthesis, with endogenous, wild-type ACP molecules. After infection with a helper phage, bacteria produce bacteriophage that express the modified ACP-coat protein fusions, carrying a fatty acid, on the bacteriophage coat protein.

Alternatively, the amount of modified ACP-coat protein fusion may be increased by using a plasmid to express the coat protein fusion prior to bacteriophage infection. After the desired amount of ACP-coat protein fusion is modified with a fatty acid, E. coli cells are infected with bacteriophage. This method is analogous to the one using a helper phage because both approaches lead to the overproduction of a modified ACP-coat protein fusion prior to bacteriophage assembly.

Synthesis and Display of Novel Fatty Acids

To increase the amount and variety of unsaturated and/or saturated fatty acids synthesized by E. coli cells, one or more nucleic acids that encode proteins involved in fatty acid synthesis may be mutated to generate proteins with altered substrate specificity or catalytic efficiency (Example 7). Alternatively, heterologous fatty acid synthases may be expressed in E. coli cells. Cells are then infected with a modified bacteriophage containing the coat protein fusion described above using standard procedures.

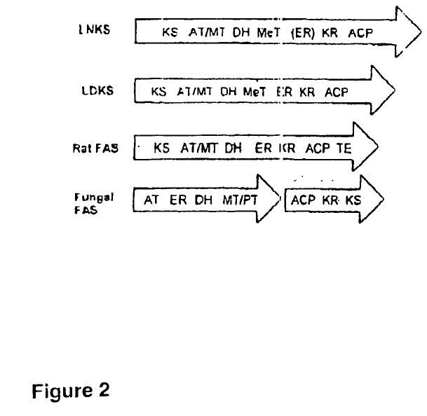

Alternatively, the above method may be performed using an acyl carrier protein domain (ACP-domain) from a multidomain enzyme as the display peptide in the protein fusion instead of an ACP FIG. 2 illustrates one example of a multidomain fatty acid synthase.

Selection and Identification of Fatty Acids that Bind a Target Molecule

Bacteriophage generated from any of the above methods that express fatty acids on their surface may be collected and purified using standard procedures. For example, bacteriophage displaying a fatty acid that binds a target molecule of interest may be selected using the immobilized target molecule in a standard column chromatography, magnetic bead purification, or panning procedure (see, for example, Ausubel et al., supra). The isolated bacteriophage may be treated with Factor Xa to cleave the linker connecting the ACP-fatty acid complexes to the coat protein on the surface of the bacteriophage. Bacteriophage are then removed by PEG precipitation. To separate fatty acids from ACP molecules, thioester linkages between fatty acids and ACP molecules are cleaved by treatment with hydroxylamine at pH 6.5 (Rosenfeld et al., Anal. Biochem. 64:221-228 1975), with sodium borohydride (Barron et al., Anal. Biochem. 40:1742-1744 1968), or with a non-specific thioesterase or esterase. Identification of the recovered fatty acids may be performed by mass spectrometry or NMR analysis.

### EXAMPLE 5

Display of Small Groups Involved in Fatty Acid Synthesis on T7 Bacteriophage

Other organic molecules can be displayed via a covalent attachment to a protein on the surface of a phage, such as a T7 bacteriophage. Small groups which are involved in fatty acid synthesis, using as a starting material acetyl-CoA and malonyl-CoA, separately were used. The reactions are summarized below:

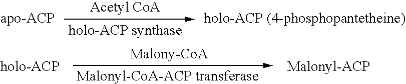

E.coli acyl carrier protein (ACP) was selected to be the support for anchoring acetyl and malonyl groups and the endogenous E. coli fatty acid machinery was used to attach malonyl and acetyl groups to ACP, which is displayed on the surface of T7-ACP. The ACP gene was fused to the C-terminus end of protein 10B of T7 (T7 select display system, Novagen). For ease of purification, a hexa-histidine tag was added to the C-terminus of ACP and the sequence (IEGR), which is recognized by the endoproteinase Factor Xa, at the amino end of ACP. BLT5615 E.coli cells were chosen as hosts for T7. These cells contain a plasmid that supplies large amounts of capsid protein, which is required for bacteriophage assembly. The promoter that regulates the production of such protein is IPTG-inducible.

Cloning of ACP

The gene coding for ACP was obtained by PCR from chromosomal DNA of E.coli strain ATCC No. 11303 by using Pfu DNA polymerase (Stratagene). This gene was amplified using a nested-PCR approach. Initially, a PCR reaction was performed using the primers ACP-FXa-FW (5′-ATC GAG GGA AGG ATG AGC ACT ATC GAA GAA CGC GTT AAG AAA AT-3′; SEQ ID NO: 49) and ACP-HIS-BK (5′-TGA TGG TGA TGA TGA TGC GCC TGG TGG CCG TTG ATG TAA TCA ATG-3′; SEQ ID NO: 50). The PCR product of this reaction was used as a template for a second PCR reaction using the primers ACP-EcoRI-FW (5′-TCA CTC GAA TTC GAT CGA GGG AAG GAT GAG CAC TAT CGA AGA ACG-3′; SEQ ID NO: 51) and ACP-HindIII-BK (5′-ATG GAT AGG AAG CTT TTA GTG ATG GTG ATG ATG ATG CGC CTG GTG-3′; SEQ ID NO: 52). The final PCR product was purified and digested with EcoRI and HindIII. This fragment was ligated with T7 EcoRI/HindIII arms (Novagen), which had already been digested with these two enzymes. Then, the ligation mixture was combined with the T7 packaging extract for in vitro packaging. Fully assembled T7 bacteriophage were diluted in LB, incubated with BLT5615 cells, and plated onto LB plates using Top LB containing IPTG. A T7 bacteriophage with the desired sequence was found and named T7-ACP.

Host Cells The strain BLT5615 (Novagen) was used as the host cell. This strain was grown in M9 minimal medium plus 0.4% glucose, 100 μM biotin, 1 mM thiamine, 1 mM MgSO₄, 0.1 mM CaCl₂, 100 μg/ml ampicillin.

ACP Production

Display of Acetyl- and Malonyl-Containing Compounds

BLT5615 cells were grown in minimal media, as described above, to minimize the amount of β-alanine, which is a precursor of CoA, within the cells. This was done to ensure the attachment of radiolabeled acetyl and malonyl groups to ACP. All experiments were started with 25 ml of minimal media (see above) containing 10 μl of BLT5615 cells grown to the beginning of log phase and stored at 4° C. When the 25 ml reached OD₆₀₀ ˜0.05, an aliquot of 5 ml was incubated with 50 μl of [2-¹⁴C]Malonyl-CoA (52 Ci/mol, 20 μCi/ml; Amersham) and, separately, another 5 ml aliquot was incubated with 100 μl of [³H]Acetyl-CoA (230 Ci/mol, 50 μCi/ml; Amersham). When cells reached OD₆₀₀=0.5, IPTG was added to a final concentration of 1 mM. Then, 30 minutes later, approximately 2-3×10³ T7-ACP molecules were added to each container. Growth was continued for about 3 hours until cells lysed. Then, the cell lysate was removed by centrifugation and the supernatant, containing T7-ACP molecules, was saved.

Purification of Radiolabeled ACP Using a Nickel Column

The saved supernatant, containing radiolabeled T7-ACP molecules, was incubated with ⅙ vol of supernatant of 20% PEG 8000 in 2.5 M NaCl. Samples were mixed and incubated for 30 minutes on ice. Then, the mixtures were centrifuged at 12,000 rpm for 15 minutes to precipitate T7-ACP molecules. Pellets, derived from 2 ml of T7-ACP, were resuspended in 400 μl of 2 mM CaCl₂, 100 mM NaCl, 20 mM Tris-HCl pH 8.0 plus 2 μg of Factor Xa. This mixture was incubated for 16 hoursr at room temperature (˜22° C.) to cleave ACP from the 10B coat protein. The mixture was loaded onto a minicolumn containing a disk of nickel-agarose (Pierce) that swells to 200 μl of binding matrix. The column was equilibrated with 150 mM NaCl, 20 mM Tris-HCl pH 7.4 and the sample was loaded. The column was washed five times with 400 μl of 150 mM NaCl, 20 mM Tris-HCl pH 7.4, and ACP was eluted with three washes of 400 μl of with 50 mM EDTA, 150 mM NaCl, 20 mM Tris-HCl pH 7.4.

Results

The data below in Table VI indicates the amount of counts detected in the flow through of the column used to purify ACP from T7-ACP molecules grown in the presence of [2-¹⁴C]Malonyl-CoA and [³H]Acetyl-CoA, separately.

| | Sample incubated | Sample incubated |
|---|---|---|
| | with Malonyl-CoA | with Acetyl-CoA |
| | (dpm) | (dpm) |
| Sample | 3550 | 6389.7 |
| Wash # 1 | 2246.6 | 3700 |
| Wash # 2 | 245.2 | 788.5 |
| Wash # 3 | 101.2 | 287.4 |
| Wash # 4 | 80.9 | 156.3 |
| Wash # 5 | 110.6 | 100 |
| Elution | 479.7 | 307.5 |

In the case of the sample incubated with malonyl-CoA, the eluted sample is equivalent to 4.16 pmoles or 2.5×10¹² T7-ACP molecules. Since the T7-ACP titer is at most 1×10¹¹/ml, this indicates that there is approximately an average of 12.5 malonyl groups attached to ACP. In the case of the sample incubated with acetyl-CoA, the eluted sample is equivalent to 0.6 pmoles or 3.6×10¹¹ T7-ACP molecules. Since the T7-ACP titer is at most 1×10¹¹/ml, this implies that there are on average 1.8 acetyl groups attached to ACP

Size-Exclusion Analysis of Radiolabeled ACP

In this experiment, size-exclusion filtration was used to determine if ACP displayed on the surface of T7-ACP molecules, which was grown in the presence of [2-¹⁴C]Malonyl-CoA and [³H]Acetyl-CoA, separately, is radiolabeled. Accordingly, the amount of radiation in a solution containing T7-ACP molecules that flows through a 100 kDa-cutoff filtration membrane was measured. The same experiment was then repeated, but prior to the filtration, the solution was first incubated with Factor Xa. Since ACP is fused to the 10B protein of T7-ACP via a peptide recognized by Factor Xa and ACP is only 8.8 kDa, treatment with Factor Xa should release ACP from the bacteriophage surface and ACP should flow through the 100 kDa-cutoff filtration membrane. If ACP is radiolabeled, then there should be an increase in the amount of radiation that flows through the membrane. The experimental details and results are shown below.

Two ml of precipitated T7-ACP molecules was dissolved in 1 ml of 150 mM NaCl, 20 mM Tris-HCl pH 7.4 and 300 μl of T7-ACP labeled in the presence of [2-¹⁴C]Malonyl-CoA and [³H]Acetyl-CoA, separately, was digested with Factor Xa. The above samples were then filtered using a 100 kDa-cutoff filtration membrane and 300 μl of T7-ACP samples that were not treated with Factor Xa was used as a control. 200 μl of those samples was collected and the amount of radiation in the flow through was measured by scintillation counting. It is important to note that a significant fraction of the counts flowing through the membrane are derived from unattached [2-¹⁴C]Malonyl-CoA and [³H]Acetyl-CoA that was leftover in the residual volume containing the precipitated T7-ACP molecules.

Results are normalized to 2 ml of T7-ACP and background signal was already subtracted.

Results

TABLE VII

| | Sample incubated | Sample incubated |
|---|---|---|
| | with Malonyl-CoA | with Acetyl-CoA |
| | (dpm) | (dpm) |
| Without Factor Xa | 3005.2 | 9887.9 |
| With Factor Xa | 3347.2 | 10037.9 |
| Radiolabeled ACP | 342 | 150 |

These results show that ACP has been radiolabeled, separately, with [2-¹⁴C]Malonyl-CoA and [³H]Acetyl-CoA. This data suggests that there are an average of 8.9 and 0.9 radiolabeled malonyl and acetyl groups attached to ACP, respectively, assuming a titer of 1×10¹¹ T7-ACP/ml (Table VII above).

Non-Denaturing PAGE Analysis of Radiolabeled Material Attached to ACP

A 15% non-denaturing gel (Tris-HCl pH 8.0) analysis was performed to examine if ACP displays radiolabeled small groups involved in fatty acid synthesis. To release ACP from the bacteriophage coat, T7-ACP molecules labeled with [2-¹⁴C]Malonyl-CoA and [³H]Acetyl-CoA, separately, were incubated with 2 μg of FactorXa for 14 hrs in a 200 μl buffer containing 50 mM NaCl, 2 mM CaCl₂ 20 mM Tris-HCl pH 8.0. The samples were then concentrated to 18 μl. After the addition of buffer, samples were run for approximately 1.25 hours at 10 V/cm. Following the electrophoretic run, the gel was exposed to an X-ray film for 42 hrs.

Results

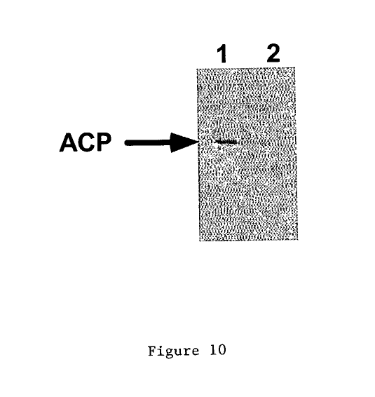

As seen below in FIG. 10, the autoradiogram shows a single band in the lane that was loaded with T7-ACP, grown in the presence of [2-¹⁴C]Malonyl-ACP and digested with Factor Xa. No band was detected in the lane loaded with T7-ACP, grown in the presence of [³H]Malonyl-CoA, and digested with Factor Xa. Thus, the non-denaturing gel confirms that[2-¹⁴C]Malonyl has been attached to ACP. This is supported by the fact that the whole T7-ACP bacteriophage cannot penetrate the gel. As discussed above, the endoproteinase Factor Xa recognizes the sequence IEGR between the 10B protein and ACP, and therefore, digestion with Factor Xa releases ACP from the bacteriophage surface. Since protein 10B remains attached to the surface of the bacteriophage and does not enter the gel, Factor Xa releases ACP with the attached [2-¹⁴C]Malonyl groups is the only protein able to penetrate the gel.

Summary

In summary, the experimental results have shown that it is possible to display on the surface of T7-ACP, small groups involved in fatty acid synthesis covalently linked to ACP. Both the nickel column purification and the filtration through a 100 kDa-cutoff membrane also suggest that ACP, on the surface of T7-ACP, is labeled, separately, with malonyl and acetyl groups derived from [2-¹⁴C]Malonyl-CoA and [³H]Acetyl-CoA, respectively. Based on the number of counts detected on ACP and the titer of T7-ACP, our data suggests that there is on average 0.9-1.8 acetyl groups derived from [³ H]Acetyl-CoA attached to ACP, and 8.9-12.5 malonyl goups derived from [2-¹⁴C]Malonyl-CoA attached, separately, to ACP. In addition, the electrophoretic gel analysis further demonstrates that ACP has been modified in vivo by the endogenous E.coli fatty acid synthesis machinery and that ACP is able to display small groups involved in fatty acid synthesis using [2-¹⁴C]Malonyl-CoA as a precursor.

### EXAMPLE 6

Display of Fatty Acids on the Surface of Yeast

Expression and Display of Fatty Acids

For the display of fatty acids on the surface of yeast, a nucleic acid encoding an ACP gene (e.g., E. coli ACP or a yeast ACP) is fused to a nucleic acid encoding all or part of the yeast Aga2p protein subunit of α-agglutinin, which is a surface protein involved in cell adhesion (Schreuder et al., Trends Biotechnol. 14:115-120, 1996). An expression system similar to pCT302 may be used for insertion of the ACP nucleic acid in-frame with the yeast Aga2p nucleic acid (Boder et al., Methods Enzymol. 328:430-444, 2000). These two nucleic acids are preferably connected with a linker encoding a recognition sequence that is cleaved by Factor Xa.

Yeast cells (e.g., Saccharomyces cerevisiae strain EBY 100) are transformed with the vector using standard methods and grown in the presence of the appropriate antibiotic (e.g., ampicillin or tetracycline) (Boder et al., supra; Cereghino et al., Curr. Opin. Biotechnol. 10:422-427, 1999). This method may also be used with any commercially available yeast expression systems such as YES, pTEF1, or spECTRA systems (Invitrogen). Examples of yeast strains that may be used in these methods include those that utilize methanol (e.g., Candida boidinii, Hansenula polymorpha, Pichia methanolica, or Pichia pastoris), lactose (e.g., Kluyveromyces lactis), starch- (e.g., Schwanniomyces occidentalis), xylose (e.g., Pichia stipitis), and alkanes and fatty acids (e.g., Yarrowia lipolytica).

The ACP protein fusion is modified by endogenous yeast enzymes. In particular, the 4′-phosphopantetheine cofactor is added to a serine in ACP by endogenous ACP-synthase, and a fatty acid is added to the free sulfhydryl group of the cofactor by endogenous yeast fatty acid synthases. If it is necessary to increase the amount of protein fusion that is modified with a fatty acid, the protein fusion may be overexpressed using a vector with a stronger promoter or using a vector that is maintained at a higher copy number in the cells. Additionally, E. coli or yeast fatty acid synthase may also be overexpressed using an inducible promoter such as pLac to increase the amount of modified ACP protein fusion (Lambalot et al., supra). To overexpress the fatty acid synthase, a vector containing the fatty acid synthase nucleic acid is transformed into yeast. As described above, pantothenate (e.g., 1-100 mM) and didemnin B (e.g., 1-100 mM) may be added to increase the amount of ACP protein fusion that remains modified with a fatty acid.

If the expression of the coat protein fusion, ACP synthase, or fatty acid synthase is harmful to yeast, the level of expression may be reduced to a suitable level by introducing an amber codon prior to the sequence encoding the protein fusion and using an amber suppression yeast strain (Christmann et al., Protein Eng. 12:797-806, 1999). Alternatively, the expression level may be controlled using plasmids maintained at the desired copy number within the cell (Daugherty et al., Protein Eng. 12:613, 1999).

Synthesis and Display of Novel Fatty Acids

To increase the amount and variety of fatty acids synthesized by yeast, one or more endogenous fatty acid synthase nucleic acids may be mutated using standard methods such as those described in Example 7 to generate synthases with altered substrate specificity or catalytic efficiency. Alternatively, heterologous fatty acid synthases may be expressed in yeast.

Selection and Identification of Fatty Acids that Bind a Target Molecule

Yeast cells generated from any of the above methods that express fatty acids may be selected and purified using standard procedures, such as those described herein. Yeast cells are treated with Factor Xa to cleave the linker in the ACP protein fusion between ACP and Aga2p. Then, yeast cells are separated from ACP-fatty acid molecules by centrifugation. Soluble proteins from the supernatant are then treated with hydroxylamine at pH 6.5 or sodium borohydride as described above to cleave the thioester linkage and produce soluble fatty acids. The recovered fatty acids may be identified using standard mass spectrometry or NMR analysis.

### EXAMPLE 7

Display of Nonribosomally Synthesized Polypeptides and Polyketides

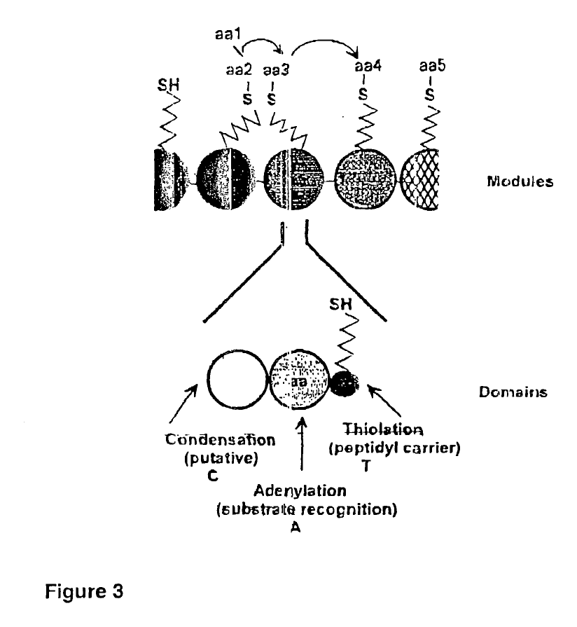

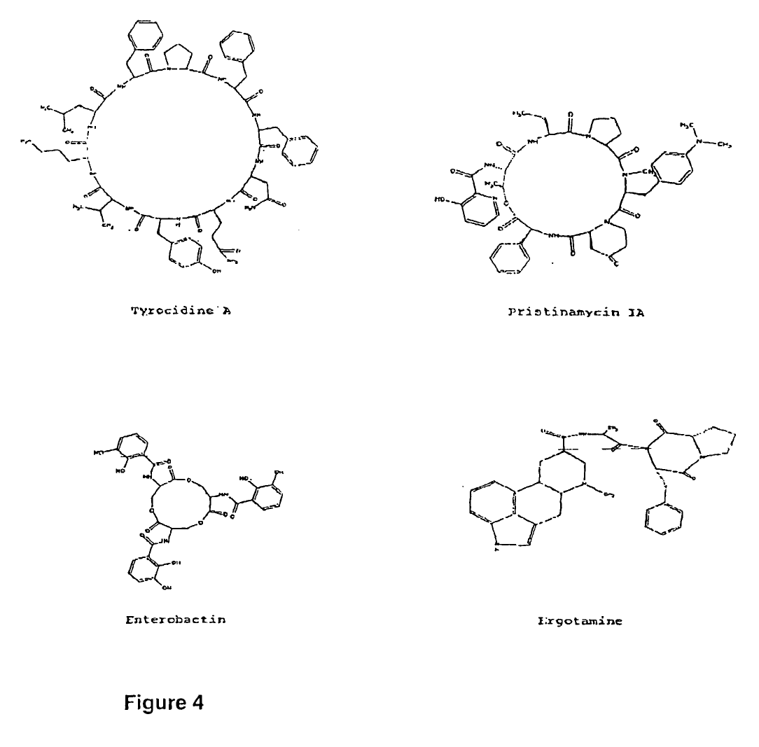

A large number of polypeptides and polyketides of medicinal and biotechnological interest are synthesized by modular enzyme complexes instead of ribosomes. Each module is responsible for incorporating one specific amino acid into a growing chain whose length is determined by the number of modular units that are present. Each module of a nonribosomal polypeptide synthase can be further subdivided into different domains (FIG. 3). The adenylation domain (A-domain) catalyzes adenylation, which leads to the activation of a cognate peptide. This activated amino acid is then covalently linked to the 4′-phosphopantetheine cofactor bound to a peptidyl carrier protein thiolation domain (T-domain). The condensation domain (C-domain) catalyzes the condensation of the amino acid linked to the T-domain with the peptidyl moieties bound to neighboring modules. In addition to these three main domains, modules may contain other domains that catalyze covalent modifications of a tethered amino acid. For example, module 4 of the Tyrocidine A synthesis pathway contains an epimerization domain (E-domain) which converts a tethered amino acid from one to another isomeric form (Mootz et al., Proc. Natl. Acad. Sci. U.S.A. 97:5848-5853, 2000). Moreover, various amino acids and amino acid analogs may be incorporated into a polypeptide, such as L-amino acids and over 300 unusual, nonproteinogenic residues [e.g., D-amino acid, β-amino acids, hydroxy acids, and N-methylated acids (FIG. 4) (von Dohren et al., Chem. Biol. 10:R273:279, 1999)].

Mechanism for the Synthesis of Tyrocidine

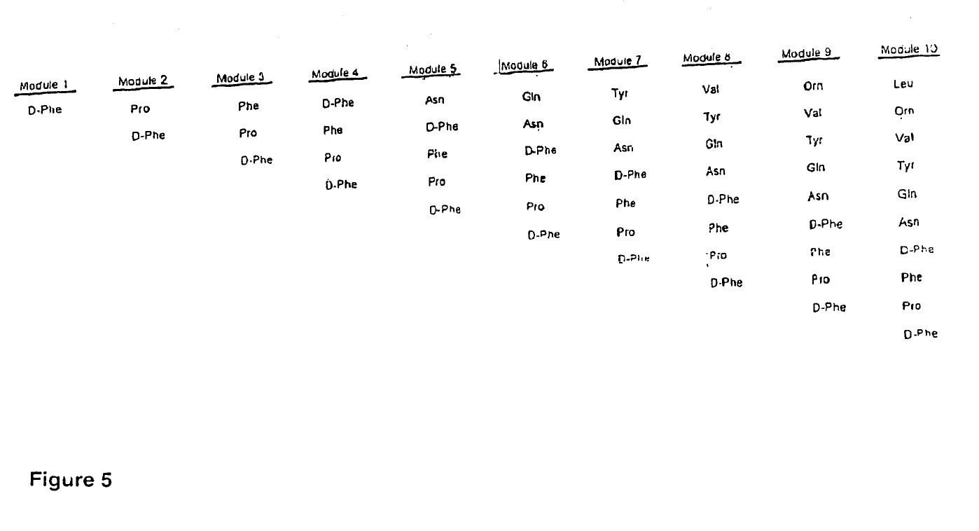

Extensive information is available regarding the independent modules responsible for the biosynthesis of many polypeptides and polyketides, such as the cyclic decapeptide antibiotic Tyrocidine A. The formation of Tyrocidine A involves three genes tycA, tycB, and tycC that encode synthases which incorporate in sequential order one, three, and six amino acid residues, respectively, into the growing polypeptide chain (Mootz and Marahiel, J. Bacteriol. 179:6843-6850, 1997) (FIG. 5).

The tycA gene encodes tyrocidine synthetase I, which includes A-, T-, and E-domains. This gene is responsible for chain initiation. The tycB gene encodes tyrocidine synthetase II, which consists of three modules that have C-, A-, and T-domains with a terminal epimerization domain at the end of the third module. The tycC gene encodes tyrocidine synthetase III, which includes six modules that have C-, A-, and T-domains with a thioesterase domain (Te) at the end of the sixth module. The Te-domain is believed to catalyze the cyclization and release of the peptide chain.

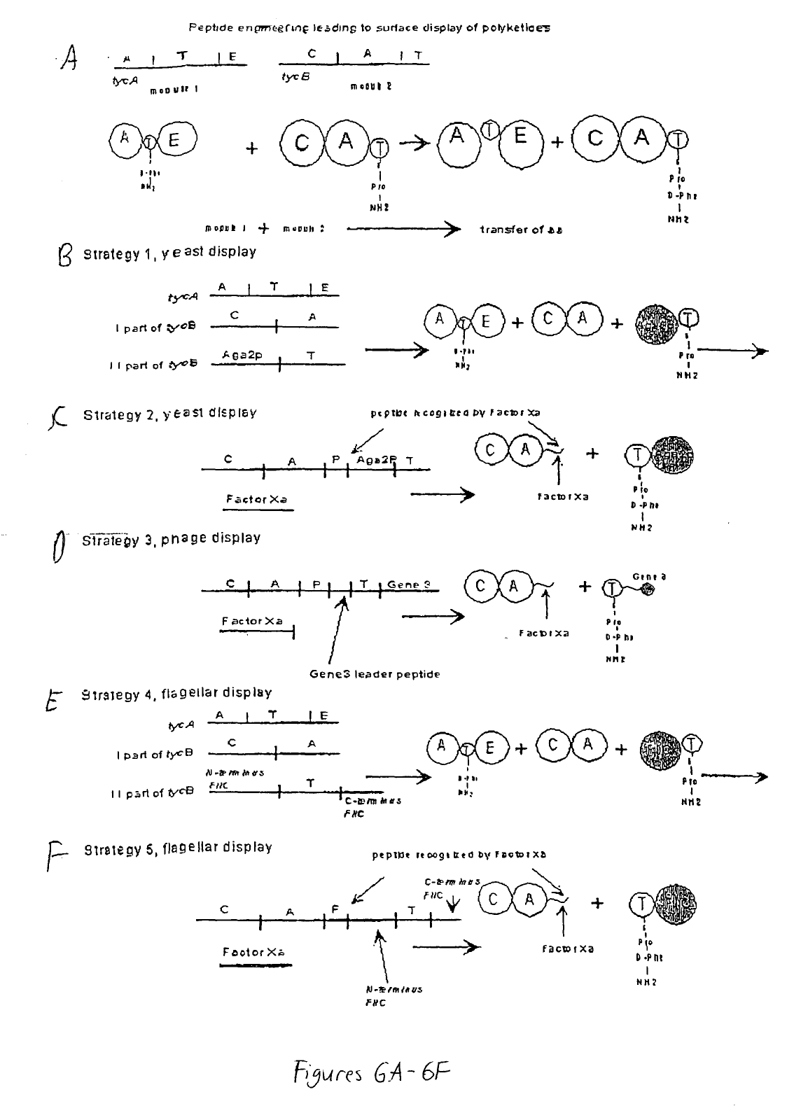

In particular, the tycA gene encodes the module responsible for the incorporation of D-Phe into Tyrocidine A. The first module of tycB is responsible for addition of L-Pro to the growing polypeptide chain. For the synthesis of the D-Phe-L-Pro dipeptide intermediate, the tycA module is covalently modified with D-Phe and the first module of tycB is covalently modified with L-Pro (FIG. 6A).

The D-Phe residue bound to the tycA module is then condensed with the nearby L-Pro residue bound to the first module of tycB, generating the D-Phe-L-Pro dipeptide bound to the first module of tycB.

Strategies for the Display of Polypeptide or Polyketide Intermediates or Full-Length Products on the Surface of Yeast, Bacteriophage, or Bacteria

(i) To display the D-Phe-L-Pro dipeptide on the surface of yeast yeast cells are transformed with a plasmid containing the tycA gene which encodes the D-Phe module and a nucleic acid encoding the first two domains of the Pro module (i.e., the C-domain and A-domains without the T-domain). The gene encoding the T-domain of the L-Pro module is fused to the carboxyl end of the Aga2p gene for the production of a protein fusion (FIG. 6B). The first two domains of the Pro module act in trans with the T-domain in the protein fusion to catalyze the same reactions that are naturally catalyzed by the intact L-Pro module. Specific recognition sequences may also be added to enhance the communication between the A- and T-domains of the L-Pro module. Thus, this strategy results in the covalent attachment of the D-Phe-L-Pro dipeptide to the T-domain in the protein fusion and the expression of this modified protein fusion on the surface of yeast.

A similar strategy may be used to display the D-Phe-L-Pro dipeptide on the T-domain, which is fused to the coat protein of a bacteriophage. In this method, the T-domain of the Pro module is fused to the pIII coat protein instead of the Aga2p yeast protein. This coat protein fusion is produced by bacteria and assembled into the bacteriophage coat protein, as described in previous examples.

(ii) To increase the amount of the L-Pro T-domain in the protein fusion that is modified with the dipeptide, the above methods may be altered to enhance communication between the A- and T-domains of the L-Pro module (Tsuji et al. Biochemistry, 40:2317-2325, 2001). To this end, the A- and T-domains are expressed by one plasmid and connected via a flexible linker, which contains a recognition sequence for the protease Factor Xa (FIG. 6C) (see, for example, Ausubel et al., supra). The nucleic acid encoding Factor Xa is placed under the control of an inducible promoter. Once the D-Phe-L-Pro dipeptide is synthesized, expression of the protease is induced so that the recombinant protein module is cleaved into a desired segment containing Aga2p-T-domain and a segment containing the C- and A-domains. The Aga2p-T-domain, which contains the covalently bound D-Phe-L-Pro dipeptide, is then transported to the surface of yeast cells.

(iii) A similar strategy is illustrated in FIG. 6D for generating a recombinant module that allows the polypeptide to be displayed on the surface of a bacteriophage. This alternative strategy minimizes the size of the insert between the A-domain and the T-domain of the L-Pro module and thus may increase the ability of the recombinant protein to synthesize the D-Phe-L-Pro dipeptide. In particular, a 2.5 kDa insert that contains the phage gene III leader sequence and the Factor Xa cleavage site is inserted between the coding sequence for the A- and T-domains of the L-Pro module. The gene III coding sequence is also added to the 3′ end of the coding sequence for the T-domain. The 2.5 kDa insert used in this methods is four-fold smaller than the insert (which encodes the Factor Xa cleavage site and Aga2p) used in the yeast display method described above. The nucleic acid encoding Factor Xa is placed under the control of an inducible promoter.

(iv) Another method for the display of the dipeptide uses an even smaller insert between the A- and T-domains of the L-Pro module. In this method, a vector is used that encodes for a recombinant protein that includes the C-, and A-domains, a four-amino acid factor Xa cleavage site, the T-domain, and a small “binding” protein that has high affinity for a “partner” protein. The partner protein is fused to the bacteriophage pIII coat protein. For example, protein kinase A (PKA) isoform alpha inhibitor can be used as the binding protein in the recombinant protein, and PKA can be used as the partner protein component of the coat protein fusion. These proteins interact with each other with high affinity (K₁=98 pM) (Wen et al., J. Biol. Chem. 270:2041-2046, 1995). Alternatively, a peptide and an antibody reactive with the peptide may be used to form a high affinity complex.

For the expression of these proteins, one or more vectors that together encode the recombinant protein module, factor Xa, and tycA, and the vector, encoding a bacteriophage with the coat protein fusion are transformed into E. coli or yeast cells. After the synthesis of the D-Phe-L-Pro dipeptide which remains bound to the T-domain in the recombinant protein module (e.g., after 30, 60, or 90 minutes), the gene encoding factor Xa is induced, and the protein product cleaves the recombinant protein module. One of the cleavage products contains the modified T-domain fused to the binding protein. The binding protein and the partner protein of the coat protein fusion associate with each other through a high affinity, non-covalent interaction. This complex is secreted into the media and displayed on the bacteriophage surface.

(v) Polypeptide intermediates and products may also be displayed using bacteria flagellar display methods. These methods are analogous to those used for yeast display except that the display peptide is fused to a bacteria flagella surface protein, such as E. coli FliC. An advantage of the flagellar display system is that thousands of copies of the flagella protein fusion are displayed. These proteins increase the affinity of the protein fusion for a target molecule and facilitate the detection of displayed polypeptides which bind the target molecule. The coding sequence for the T-domain of the Pro module (or the T-domain of any other polypeptide module) is inserted into the FliC_{H7} gene, which encodes the variable domain of the H7 flagellin (FIG. 6E). One or more vectors that together contain this nucleic acid construct, the tycA coding sequence, and a nucleic acid encoding the first two domains of the Pro module (i.e., the C-domain and A-domains without the T-domain) is expressed in E. coli JT1 strain, which has a FliC knockout mutation that prevents the expression of functional, endogenous FliC (Westerlund-Wikstrom et al., Prot. Eng. 10:1319-1326, 1997; Tanskanen et al., Appl. Environ. Microbiol. 66:4152-4156, 2000). Because the central, highly variable region of FliC forms a surface-exposed domain that is responsible for the antigenic variability in flagella (Namba et al., Nature 342:648-654, 1989) and that tolerates large deletions and insertions without loss of flagellar polymerization (Kuwajima, J. Bacteriol. 170:3305-3309 1988), insertion of the coding sequence for the T-domain into this region of the FliC_{H7} gene results in the display of the modified T-domain on the bacterial surface.

In an alternative flagella display method, a protein fusion is generated that contains C-and A-domains in close proximity to the T-domain and thus maintains most or all of the activity of the wild-type L-Pro module. In particular, a nucleic acid encoding the C- and A-domains, and a protease cleavage site is fused to the 5′ end of the FliC_{H7} and the T-domain is inserted into the variable domain of the FliC_{H7} gene (FIG. 6F). This protein fusion and tycA are expressed in E. coli JT1 strain, resulting in the covalent attachment of the D-Phe-L-Pro dipeptide to the T-domain of the protein fusion. Then, the expression of the protease is induced to cleave the protein fusion. The cleavage product containing the modified T-domain inserted into the variable region of the FliC flagella is then transported to the surface of E. coli cells.

These flagella display methods may also be performed using flagella proteins from any other bacteria. These proteins may be expressed in E. coli (e.g., E. coli JT1 strain), other bacteria that naturally contain the corresponding flagella gene, or any other bacteria. Bacteria expressing the flagella protein fusion may also express wild-type, endogenous flagella proteins or may contain a mutation that reduces or eliminates the expression of endogenous flagella proteins. Exemplary flagella proteins useful in the invention are listed below in Table VIII.

TABLE VIII
Flagella proteins for use in protein fusions to display
compounds on the surface of bacteria

1. Probable export protein fliO (Salmonella typhimurium; accession

numbers L49021 and S78697)

2. Probable flagellar biosynthesis protein mopB (Erwinia carotovora

subsp. Atroseptica; accession number S35275)

3. Protein mopB (Pectobacterium carotovorum; accession number

CAA51475.1)

```
  4. Burkholderia pseudomallei (accession number U73848)
  5. Ralstonia solanacearum (accession number AF283285)
  6. Clostridium difficile (accession number AF095238)
  7. Salmonella enterica subsp. Enterica (accession number AF332601)
  8. Rhodobacter sphaeroides (accession number AF274346)
  9. Clostridium chauvoei (strain:Okinawa; accession number D89073)
10. Pseudomonas chlororaphis (accession number AJ297537)
11. Pseudomonas citronellolis (accession number AJ297535)
12. Pseudomonas fragi (accession number AJ297534)
13. Salmonella typhimurium (accession number M33541)
14. Pseudomonas aeruginosa (accession number L81176)
15. Riftia pachyptila endosymbiont (accession number AF105060)
16. Xenorhabdus nematophila (accession number AJ131736)
17. Burkholderia mallei (accession number AF084815)
18. Salmonella enterica subsp. enterica serovar Gallinarum (accession
```

number AF139681)

19. Salmonella enterica subsp. enterica serovar Pullorum (accession

number AF139674)

20. Pseudomonas putida (accession number AB018737)
21. Pseudomonas fluorescens (accession number AB018715)
22. Burkholderia cepacia (accession number AF011372)
23. Burkholderia thailandensis (accession number AF081500)
24. Brucella melitensis biovar Abortus (accession number AF019251)
25. Salmonella naestved (accession number D78639)
26. Shigella boydii (accession number D26165)
27. Shigella flexneri (accession number D16819)
28. Shigella sonnei (accession number D16820)
29. Bacillus sp. (accession number D10063)
30. Salmonella enteritidis (accession number M84980)

(vi) Any of the methods described above may be used to display polypeptide or polyketide intermediates containing more than two amino acids or to display full-length polypeptide or polyketide products. For these methods, the T-domain, thioesterase domain (Te-domain), or ACP-domain of the module responsible for the tethering of the last amino acid or small molecule is fused to the Aga2p gene, the bacteriophage gene III, or the FliC gene.

Non-circularized intermediates or products are displayed by remaining covalently bound to the last nonribosomal polypeptide or polyketide synthase attachment domain in the protein fusion (e.g., a thiolation or acyl carrier domain) that is expressed on the surface of viruses or cells. For the display of circularized products, a modified display system is used. During the synthesis of circularized nonribosomal peptides and/or polyketides, a growing chain is transferred through various domains until the growing chain is extended to its full-length. Then, the fully-grown chain is transferred from the protein that served as the last support during chain elongation to a thioesterase domain, where the ends of the polypeptide or polyketide are covalently linked to form a circularized product. Since the linear chain is immobilized at one end, the circularization process leads to the release of the circularized small molecule from the thioesterase domain. To avoid the undesired cleavage of the circularized product from the thioesterase domain, recombinant proteins that contain all of the synthase domains except for the thioesterase domain are expressed in bacteria to synthesize the linear product. To catalyze the circularization but not the release of the product, mutant thioesterase domains fused to surface proteins (e.g., viral coat proteins or flagella proteins) are also expressed in bacteria. Bacteria which express the desired mutant thioesterase domain catalyze the circularization but not the release of the polypeptide or polyketide product. Bacteria or bacteriophage that display the circularized small molecule covalently linked to the thioesterase domain are identified utilizing an agent, such as an antibody, against the circularized small molecule. Thus, thioesterase variants that can circularize a small molecule without leading to its immediate release can be readily identified.

For example, the full-length, circularized Tyrocidine A product can be displayed by fusing the Te-domain of the last module, which is responsible for the circularization of the decapeptide, to the Aga2p gene, the bacteriophage gene III, or the FliC gene. Because this Te-domain has also been associated with the release of Tyrocidine A from the module, the Te-domain may need to be modified so that it catalyzes the circularization step but not the hydrolysis of Tyrocidine A from the module. For example, random mutations may be introduced into the Te-domain, and the modified Te-domains may be assayed to determine to which domain the circularized tyrocidine product remains covalently bound.

Analogs of tyrocidine polypeptides or other polypeptides may be displayed using any of the methods described above. For example, the condensation, adenylation, thiolation, and thioesterase domains from other polypeptide synthases may be readily identified based on their homology to the corresponding domains from other synthases and used in the methods described herein. Examples of other nonribosomal peptides that may be displayed using these methods include yersiniabactin (Pelludat et al., J. Bacteriol. 180:538-546, 1988), mycosubtilin (Duitman et al., J. Proc. Natl. Acad. Sci. U.S.A. 96:13294-13299, 1999), fengycin (Steller, et al., J. Chem. Bio. 6:31-41, 1999) ergopeptines (Riederer et al., U. J. Biol. Chem. 271:27524-27530, 1996), bacillibactin (May et al., J. Biol. Chem. 276:7209-7217, 2001), etamycin (Schlumbohm et al., J. Biol. Chem. 265:2156-2161, 1990), and actinomycin (Pfennig et al., J. Biol. Chem. 274:12508-12516, 1999).

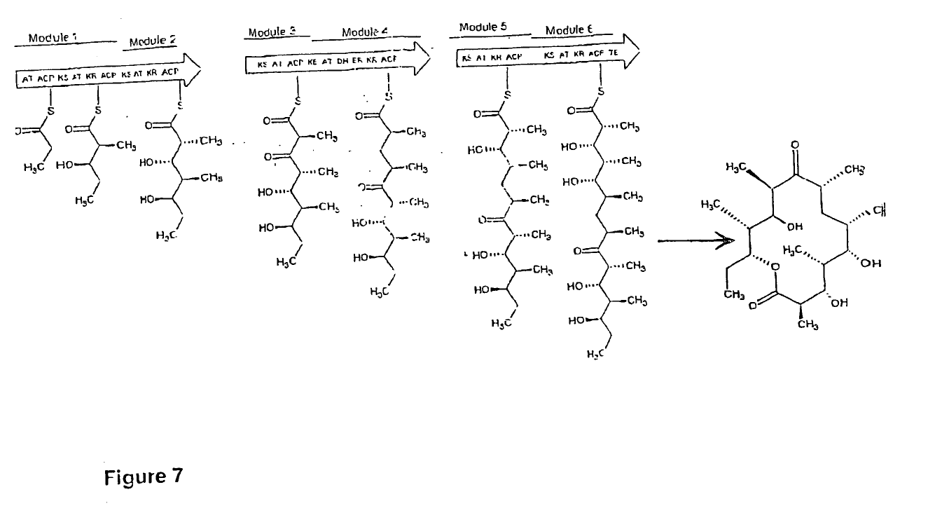

Any of the above methods may also be readily adapted for the display of novel or naturally-occurring polyketide intermediates or products. For example, a well characterized polyketide that can be displayed using these methods is 6-deoxyerythronolide B. The 6-deoxyerythronolide B polyketide synthase has six modules with different domains within each module (FIGS. 7A-7C). For example, module 1 contains a ketosynthetase (KS) domain, an acyl transferase (AT) domain, a ketoreductase (KR) domain, and an acyl carrier protein (ACP) domain. Modules 2, 5, and 6 are similar to module 1 but have different linker sequences. Other examples of polyketides include polyketides that catalyze the desaturation and elongation steps in lipid metabolism (Metz et al., Science 293:290-293, 2001). Additionally, the condensation, adenylation, thiolation, and thioesterase domains from other polyketide synthases and nonribosomal synthases may be readily identified based on their homology to the corresponding domains from other synthases and used in the methods described herein.

Novel polypeptides and polyketides may also be synthesized and displayed on the surface of yeast, bacteriophage, or cells. For example, one or more endogenous nonribosomal polypeptide synthase, polyketides synthase, or hybrid polyketide/nonribosomal peptide synthase nucleic acids may be mutated to generate synthases with altered substrate specificity or catalytic efficiency. Alternatively, one or more heterologous nonribosomal polypeptide synthases, polyketide synthases, and/or hybrid polyketide/nonribosomal peptide synthases, such as those described herein, may be expressed in the yeast or bacteria.

Selection and Identification of Displayed Polypeptides or Polyketides which Bind a Target Molecule

Bacteria, yeast cells, or bacteriophage displaying a polypeptide or polyketide which binds a target molecule may be selected using standard methods, and then the polypeptides or polyketides may be recovered from the selected bacteriophage or cells. To cleave the polypeptides or polyketides, hydroxylamine at pH 6.5 or sodium borohydride can be used to cleave the thioester linkage between the polypeptide or polyketide and the display peptide (Rosenfeld et al., supra; Barron et al., supra). The polypeptides or polyketides of interest can be identified using mass spectrometry or NMR. The polypeptides and polyketides may be tested for their ability to inhibit the growth of, or to kill, certain bacteria, such as those associated with infections in humans or animals of veterinary interest.

### EXAMPLE 8

Generation and Display of Novel Compounds

To create a variety of small molecules that are expressed on the surface of viruses or cells, endogenous or heterologous genes encoding proteins involved in the synthesis of a molecule of interest may be mutated to alter the substrate specificity or catalytic efficiency of proteins. In particular, random mutations may be introduced into the key enzymes participating in secondary metabolic pathways from different organisms. Examples of nucleic acids that may be mutated include those that encode a biotin ligase, phosphopantetheinyl transferase, fatty acid synthase, polyketide synthase, nonribosomal peptide synthase, lipoate ligase, glycosyltransferase, farnesyltransferase, or geranylgeranyltransferase. Cells with these mutated nucleic acids may be used in the methods of the present invention to generate and isolate novel molecules which bind a target molecule.

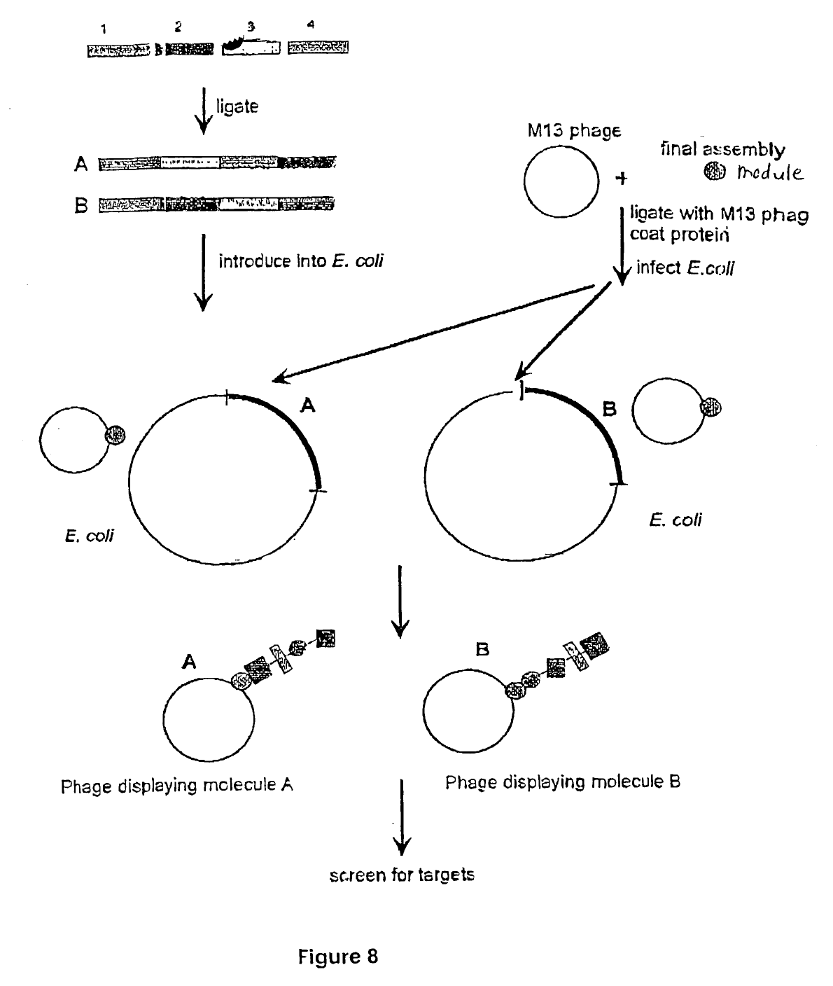

In one such mutagenesis method, one or more mutations are introduced into a nucleic acid using the polymerase chain reaction under conditions that introduce a high number of mutations (Fromant et al., Anal. Biochem. 224:347-353 1995). Other mutagenesis techniques involve in vitro homologous recombination (e.g., DNA shuffling) of polyketide, nonribosomal peptide, and/or fatty acid synthase nucleic acids from multiple organisms (FIG. 8) (Stemmer et al., Proc. Natl. Acad. Sci. U.S.A. 91:10747-10751, 1994; Coco et al., Nat. Biotech. 19:354-359, 2001).

These methods may be used to generate fatty acid synthases that produce a large variety of novel fatty acids. Exemplary fatty acid synthases that can be mutated include synthases, such as the one in Mycobacterium tuberculosis, that produce a variety of multiple methyl-branched fatty acids required for sulfolipid synthesis (Sirakova et al., J. Biol. Chem. 276:16833-16839 2001). Other fatty acid synthases, such as the Streptomyces glaucescens beta-ketoaccyl-acyl carrier protein synthase III (KASIII), initiate linear- and branched-chain fatty acid biosynthesis by catalyzing the decarboxylative condensation of malonyl-ACP with different acyl-coenzyme A (CoA) groups (Smirnova et al., J. Bacteriol. 183:2335-2342 2001). Additionally, the phospholipid fatty acid composition of the sponge Amphimedon complanata includes the following unusual phospholipids: 2-methoxy-13-methyltetradecanoic acid, 2-methoxy-14-methylpentadecanoic acid, and 2-methoxy-13-methylpentadecanoic acid). The fatty acid synthase from this sponge may be mutated to generate additional phospholipids of interest (Carballeira et al., Lipids 36:83-87, 2001).

Random mutations may also be introduced into synthetase nucleic acids to produce antibiotics with novel properties. For example, synthases that utilize different amino acids than the corresponding wild-type synthases or that catalyzed different modifications (e.g., acylation of tethered amino acids) can be generated. In addition, DNA shuffling may be used to combine synthases from multiple organisms. For this mutagenesis technique, different domains, different modules, and/or different intact polyketide synthase coding sequences may be combined.

Because fatty acid, polyketide, and nonribosomal peptide synthases are homologous, their corresponding nucleic acids may also be shuffled to generate novel compounds (Metz et al. Science 2001, 293, 290-293, 2001).

Other Embodiments

From the foregoing description, it will be apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A method for selecting a small molecule which binds a target molecule, said method comprising:
(a) expressing in a population of cells a protein fusion comprising a surface protein covalently linked to a display peptide, said expression being carried out under conditions that allow said display peptide to be modified in said cells with a small molecule other than biotin and the display of said protein fusion on the surface of said cells; wherein said small molecule
(i) is covalently bound to a side-chain of an amino acid in said display peptide;
(ii) comprises an unnatural amino acid; or
(iii) has a molecular weight less than 4,000 daltons and comprises either an unnatural amino acid or a moiety other than an amino acid;
(b) contacting said cells with said target molecule; and
(c) selecting said cells which bind said target molecule, thereby selecting said small molecules which bind said target molecule.

2. A method for selecting a posttranslational modification which binds a target molecule, said method comprising:
(a) expressing in a population of cells a protein fusion comprising a surface protein covalently linked to a display peptide, said expression being carried out under conditions that allow the posttranslational modification in said cells of said display peptide and the display of said protein fusion on the surface of said cells, wherein said posttranslational modification is not biotin;
(b) contacting said cells with said target molecule; and
(c) selecting said cells which bind said target molecule, thereby selecting said posttranslational modifications which bind said target molecule.

3. The method of claim 1 or 2, further comprising culturing said selected cells under conditions that permit cell proliferation, thereby generating additional cells which express said small molecules or said posttranslational modifications.

4. The method of claim 1 or 2, wherein said cells are bacteria.

5. The method of claim 4, wherein said bacteria are E. coli.

6. The method of claim 4, wherein said surface protein is a flagella protein.

7. The method of claim 1 or 2, wherein said cells are yeast.

8. The method of claim 7, wherein said yeast are S. cerevisiae.

9. The method of claim 7, wherein said surface protein is a receptor.

10. The method of claim 1 or 2, wherein said cells encode at least one of the proteins required for the synthesis of said small molecules or said posttransional modifications.

11. The method of claim 1 or 2, wherein, in step (b), said target molecule is immobilized.

12. The method of claim 1 or 2, further comprising recovering a compound comprising a moiety from said small molecule or comprising a moiety from said posttranslational modification.

13. The method of claim 1 or 2, further comprising repeating steps (a), (b), and (c).

14. The method of claim 1 or 2, further comprising mutating a nucleic acid in said cells prior to step (a).

15. The method of claim 1 or 2, wherein said small molecule or said posttranslational modification is a biotin analog, lipid, phosphopantetheine group, carbohydrate, prosthetic group, vitamin, ketone, carboxylic acid, alkaloid, terpene, polyketide, or polypeptide.

16. A cell expressing on its surface a protein comprising a surface protein covalently linked to a display peptide that is modified by the addition of a small molecule, wherein said small molecule is a biotin analog, phosphopantetheine, prosthetic group other than biotin, ketone, terpene, alkaloid, polyketide, palmitoyl group, myristoyl group, farnesyl group, geranylgeranyl group, lipoyl group, arachidonic acid, steroid, chondroitin sulfate, heparan sulfate, keratan sulfate, or a molecule comprising an unnatural amino acid.

17. A cell expressing on its surface a protein comprising a surface protein covalently linked to a display peptide that is modified by the addition of a small molecule, wherein said small molecule
(i) is covalently bound to a side-chain of an amino acid in said display peptide;
(ii) comprises an unnatural amino acid; or
(iii) has a molecular weight less than 4,000 daltons and comprises either an unnatural amino acid or a moiety other than an amino acid; and
wherein said cell comprises a mutated or heterologous nucleic acid that encodes a protein required for the synthesis of said small molecule.

18. A cell expressing on its surface a protein comprising a surface protein covalently linked to a display peptide that is modified by the addition of a small molecule other than biotin, wherein said small molecule
(i) is covalently bound to a side-chain of an amino acid in said display peptide;
(ii) comprises an unnatural amino acid; or
(iii) has a molecular weight less than 4,000 daltons and comprises either an unnatural amino acid or a moiety other than an amino acid; and
wherein said cell comprises a nucleic acid that encodes a protein required for the synthesis of said small molecule.

19. A cell expressing on its surface a protein fusion comprising a surface protein covalently linked to a posttranslationally modified display peptide, wherein said posttranslational modification is a biotin analog, phosphopantetheine, prosthetic group other than biotin, ketone, terpene, alkaloid, polyketide, palmitoyl group, myristoyl group, farnesyl group, geranylgeranyl group, lipoyl group, arachidonic acid, steroid, chondroitin sulfate, heparan sulfate, keratan sulfate, or molecule comprising an unnatural amino acid.

20. A cell expressing on its surface a protein fusion comprising a surface protein covalently linked to a posttranslationally modified display peptide, wherein said cell comprises a nucleic acid that encodes a protein required for the synthesis of said posttranslational modification; wherein said posttranslational modification is not biotin.

21. A cell expressing on its surface a protein fusion comprising a surface protein covalently linked to a display peptide, wherein a lipid, polyketide, or polypeptide is covalently bound to a phosphopantetheinylated amino acid in said display peptide.

22. The cell of any one of claims 16-21, wherein said cell is a bacteria.

23. The cell of claim 22, wherein said bacteria is E. coli.

24. The cell of claim 22, wherein said surface protein is a flagella protein.

25. The cell of any one of claims 16-21, wherein said cell is yeast.

26. The cell of claim 25, wherein said yeast is S. cerevisiae.

27. The cell of claim 25, wherein said surface protein is a receptor.

28. The cell of claim 17, 18, 20, or 21, wherein said nucleic acid encodes a biotin ligase, phosphopantetheinyl transferase, fatty acid synthase, polyketide synthase, nonribosomal peptide synthase, lipoate ligase, glycosyltransferase, farnesyltransferase, or geranylgeranyltransferase.

29. The cell of claim 17, 18, 20, or 21, wherein said display peptide is modified by the addition of a biotin analog, lipid, phosphopantetheine, carbohydrate, prosthetic group, vitamin, ketone, carboxylic acid, terpene, alkaloid, polyketide, or polypeptide.

30. A method for selecting a small molecule which binds a target molecule, said method comprising:
(a) expressing in a population of cells a protein fusion comprising a surface protein covalently linked to a display peptide, said expression being carried out under conditions that allow said display peptide to be modified in said cells with a small molecule other than biotin and the display of said protein fusion on the surface of viruses released from said cells infected with said virus; wherein said small molecule
(i) is covalently bound to a side-chain of an amino acid in said display peptide;
(ii) comprises an unnatural amino acid; or
(iii) has a molecular weight less than 4,000 daltons and comprises either an unnatural amino acid or a moiety other than an amino acid;
(b) contacting said viruses with said target molecule; and
(c) selecting said viruses which bind said target molecule, thereby selecting said small molecules which bind said target molecule.

31. A method for selecting a posttranslational modification which binds a target molecule, said method comprising:
(a) expressing in a population of cells a protein fusion comprising a surface protein covalently linked to a display peptide, said expression being carried out under conditions that allow the posttranslational modification in said cells of said display peptide and the display of said protein fusion on the surface of viruses released from said cells infected with said virus, wherein said posttranslational modification is not biotin;
(b) contacting said viruses with said target molecule; and
(c) selecting said viruses which bind said target molecule, thereby selecting said posttranslational modifications which bind said target molecule.

32. A virus expressing on its surface a protein fusion comprising a surface protein covalently linked to a display peptide that is modified by the addition of a small molecule, wherein said small molecule is a biotin analog, phosphopantetheine, prosthetic group other than biotin, ketone, terpene, alkaloid, polyketide, palmitoyl group, myristoyl group, farnesyl group, geranylgeranyl group, lipoyl group, arachidonic acid, steroid, chondroitin sulfate, heparan sulfate, keratan sulfate, or a molecule comprising an unnatural amino acid.

33. A virus expressing on its surface a protein fusion comprising a surface protein covalently linked to a display peptide that is modified by the addition of a small molecule, wherein said small molecule
(i) is covalently bound to a side-chain of an amino acid in said display peptide;
(ii) comprises an unnatural amino acid; or
(iii) has a molecular weight less than 4,000 daltons and comprises either an unnatural amino acid or a moiety other than an amino acid; and
wherein a nucleic acid of said virus encodes a protein required for the synthesis of said small molecule.
